(19) United States
(12) Patent Application Publication
(10) Pub. No.: US 2023/0417654 A1
(43) Pub. Date: Dec. 28, 2023

(54) ULTRAHIGH-SPEED MULTI-PARAMETRIC PHOTOACOUSTIC MICROSCOPY BASED ON A THIN-FILM OPTICAL-ACOUSTIC COMBINER

(71) Applicant: Washington University, St. Louis, MO (US)

(72) Inventors: Song Hu, St. Louis, MO (US); Fenghe Zhong, St. Louis, MO (US); Yifeng Zhou, St. Louis, MO (US)

(73) Assignee: Washington University, St. Louis, MO (US)

(21) Appl. No.: 18/310,412

(22) Filed: May 1, 2023

Related U.S. Application Data
(60) Provisional application No. 63/336,891, filed on Apr. 29, 2022.
(60) Provisional application No. 63/336,690, filed on Apr. 29, 2022.

(51) Int. Cl. G01N 21/17; G01N 29/46

(52) U.S. Cl. CPC G01N 21/1702; G01N 29/46; G01N 2021/1704; G01N 2291/02475

(57) ABSTRACT

Methods and systems for ultrahigh-speed multi-parametric photoacoustic microscopy that include an optical-acoustic combiner (OAC) configured to reflect the laser pulses into the sample and to transmit the photoacoustic signals to the transducer are disclosed.

### CROSS-REFERENCE TO RELATED APPLICATIONS

This application claims priority from U.S. Provisional Application Ser. No. 63/336,891 filed on Apr. 29, 2022, which is incorporated herein by reference in its entirety. This application also claims priority from U.S. Provisional Application Ser. No. 63/336,690 filed on Apr. 29, 2022, which is incorporated herein by reference in its entirety.

### STATEMENT REGARDING FEDERALLY SPONSORED RESEARCH OR DEVELOPMENT

This invention was made with government support under CBET2023988 awarded by the National Science Foundation and under NS099261 and NS120481 awarded by the National Institutes of Health. The government has certain rights in the invention.

### MATERIAL INCORPORATED-BY-REFERENCE

Not applicable.

### FIELD OF THE INVENTION

The present disclosure generally relates to methods and systems for ultrahigh-speed multi-parametric photoacoustic microscopy.

### BACKGROUND OF THE INVENTION

Photoacoustic microscopy (PAM) is a powerful tool in small-animal research. Relying on the optical absorption contrast of blood hemoglobin, PAM can visualize the vasculature and its changes in response to stimulations or under disease conditions in a label-free manner. Enabling simultaneous imaging of blood perfusion, oxygenation (sO₂), and flow speed at the single-capillary resolution, multi-parametric PAM has shed new light on the hemodynamic and metabolic bases of a wide range of diseases. However, multi-parametric PAM uses correlation-based flow measurement, which requires dense A-line sampling and thus limits the B-scan rate. This limitation impedes its application in studying rapid hemodynamics. Recently, several methods have been developed to increase PAM's scanning speed. Based on water-proofing hexagon-mirror or micro-electro-mechanical systems (MEMS), the B-scan rate of PAM can be significantly increased to 900 Hz. Unfortunately, they are not applicable for correlation-based flow quantification due to the large A-line interval. Another method takes advantage of the relaxed ultrasonic focus compared to the tight optical focus in PAM. By rapidly steering the optical spot within the ultrasonic focal zone (focal diameter: 40 μm), multiple B-scans can be simultaneously acquired to improve the imaging speed. Although promising, the imaging speed of the optical-mechanical hybrid scan-based multi-parametric PAM system is still limited by the ultrasonic focal zone of the transducer. Relaxing the focus of the spherically focused transducer will allow further improvement of the imaging speed, which however is at the expense of system sensitivity.

Capable of structural, functional, molecular, and metabolic imaging with high spatial resolution in vivo, photoacoustic microscopy (PAM) is an emerging tool in biomedical research. Further, recent advances in multi-parametric acquisition and analysis make PAM uniquely capable of simultaneously mapping the total concentration of hemoglobin (C_{Hb}), oxygen saturation of hemoglobin (sO₂), and blood flow speed. However, the dense sampling (i.e., small scanning step size) required for blood flow quantification significantly limits the imaging speed of multi-parametric PAM, preventing dynamic assessments of microvascular function and tissue oxygen metabolism.

Currently, in multi-parametric PAM, the blood flow speed is quantified by analyzing the flow-induced decorrelation between adjacent A-line signals. Although this method shows robust performance in measuring flow speeds across a wide range that is physiologically relevant (i.e., 0.18-21 mm/s) and in blood vessels of different diameters, dense sampling (i.e., 0.5 μm step size) is required to accurately extract the correlation decay constant. As a result, the B-scan rate is limited to ˜1 mm/s, which prevents the use of existing high-speed acquisition methods developed for PAM and sets up a barrier to improving the speed of multi-parametric PAM. Although other flow measurement methods have been developed or adopted for PAM, they have important limitations. For example, dynamically tracking the movement of individual blood cells is widely used for measuring microvascular flow; however, this method is not readily applicable to flow quantification in large vessels where blood cells do not traverse in single files. Dual-pulse photoacoustic flowmetry based on the Grüneisen relaxation effect shows the promise of single-shot-based flow measurements; however, an excessive average (i.e., 100 times) is required, which significantly limits the imaging speed.

### SUMMARY OF THE INVENTION

Among the various aspects of the present disclosure is the provision of methods and systems for ultrahigh-speed multi-parametric photoacoustic microscopy that include an optical-acoustic combiner (OAC) configured to reflect the laser pulses into the sample and to transmit the photoacoustic signals to the transducer.

In various aspects, a reflection-mode, ultra-high-speed, multi-parametric photoacoustic microscopy (PAM) system that includes a high-repetition-rate pulsed laser configured to produce laser pulses at first and second pulse wavelengths, a high-speed resonant galvanometer configured to scan the laser pulses in an optical scanning pattern, a cylindrically focused transducer configured to detect photoacoustic signals produced by a sample in response to the laser pulses, and an optical-acoustic combiner (OAC) configured to reflect the laser pulses into the sample and to transmit the photoacoustic signals to the transducer. In some aspects, the OAC includes a base layer, a reflecting layer formed on the base layer, and a protective layer formed on the reflecting layer opposite the base layer. In some aspects, the photoacoustic signals are ultrasound pulses. In some aspects, the base layer is acoustically matched to a coupling medium configured to acoustically couple the sample to the transducer. In some aspects, the coupling medium is water. In some aspects, the base layer includes acrylic with a density of about 1.18 g/cm³, an acoustic velocity of about 2.8×103 m/s, and a thickness of about 100 μm. In some aspects, the reflecting and protective layers each comprise a thickness much less than an ultrasonic wavelength of the photoacoustic signals. In some aspects, the reflecting layer includes aluminum with a thickness of about 250 nm. In some aspects, the protective layer includes SiO₂ with a thickness of about 190 nm.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a schematic of dual-contrast PAM. The boxed inset illustrates the laser excitation scheme designed for simultaneous dichroism and multi-parametric PAM. AOM, acousto-optic modulator; FC, fiber coupler; PM-SMF, polarization-maintaining single-mode optical fiber; BPF, bandpass filter; DM, dichroic mirror; EOM, electro-optic modulator; OL, objective lens; CL, correction lens; UT, ultrasonic transducer; WT, water tank. DAQ, data acquisition. H/V pol, horizontal/vertical polarization.

FIG. 2A is an amplitude-based PAM image of a brain section acquired with horizontally polarized 532 nm light.

FIG. 2B is a dichroism-based PAM image of the brain section from FIG. 2A.

FIG. 2C is a confocal fluorescence image of the brain section from FIGS. 2A and B. Scale bars: 200 μm.

FIG. 2D is an amplitude-based PAM image of a dissected mouse brain acquired with horizontally polarized 532 nm light. The white and cyan arrows show two representative vessels with and without amyloid deposits in the wall, respectively. The green arrows show a plaque-like structure in the amplitude-based PAM image which, however, is shown not to be a plaque in the dichroism and fluorescence images.

FIG. 2E is a dichroism-based PAM image of the dissected mouse brain from FIG. 2D. The white and cyan arrows show two representative vessels with and without amyloid deposits in the wall, respectively. The green arrows show a plaque-like structure in the amplitude-based PAM image which, however, is shown not to be a plaque in the dichroism and fluorescence images.

FIG. 2F is a confocal fluorescence image of the dissected mouse brain from FIGS. 2D and E. The white and cyan arrows show two representative vessels with and without amyloid deposits in the wall, respectively. The green arrows show a plaque-like structure in the amplitude-based PAM image which, however, is shown not to be a plaque in the dichroism and fluorescence images. Scale bars: 200 μm.

FIG. 3A is a normalized photoacoustic amplitude PAM image of a 10-month-old APP/PS 1 mouse brain in vivo, including the cerebrovascular structure.

FIG. 3B is a PAM image of a 10-month-oldAPP/PS 1 mouse brain in vivo from the same region of interest as the image seen in FIG. 3A, including the cerebrovascular structure and C_{Hb}.

FIG. 3C is a PAM image of a 10-month-oldAPP/PS 1 mouse brain in vivo from the same region of interest as the images seen in FIGS. 3A and B, including the cerebrovascular structure and sO₂.

FIG. 3D is a PAM image of a 10-month-oldAPP/PS 1 mouse brain in vivo from the same region of interest as the image seen in FIG. 3A-C, including the cerebrovascular structure and blood flow speed. Scale bars: 200 μm.

FIG. 3E is a photo of the same region imaged in FIG. 3A-D taken by a wide-field microscope.

FIG. 3F is a dichroism PAM image of the same region imaged in FIGS. 3A, 3B, 3C, 3D, and 3E showing the distribution of CR-stained amyloid plaques and deposits in the vessel wall.

FIG. 3G is a close-up image of the boxed area in FIG. 3F. Scale bars: 200 μm.

FIG. 3H is a confocal image of the boxed area in FIG. 3G. Scale bars: 200 μm.

FIG. 4 is a schematic of the PAM system used in the present disclosure. When the AOM is off, all light will go through the 0^{th} order and be coupled into the PM-SMF for the SRS effect. When the AOM is on, most of the light (˜80%) will be diffracted to the 1^{st} order without wavelength conversion. In the meanwhile, the residual light on the 0^{th} order is too weak to excite the SRS effect so all light will be completely blocked by the BPF. AOM: acousto-optic modulator; EOM: electro-optic modulator; PBS: polarizing beam splitter; PM-SMF: polarization-maintaining single mode fiber; BPF: bandpass filter; DM: dichroic mirror.

FIG. 5A is a system schematic of the configuration of the high-speed wide-field multi-parametric PAM and scanning mechanism. HWP, half-wave plate; EOM, electro-optic modulator; PBS, polarizing beam splitter; PM-SMF, polarization-maintaining single-mode fiber; BPF, band-pass filter; DM, dichroic mirror; PD, photodetector; GM, galvanometer; UT, ultrasound transducer; PF, polymer film; Objective, f=60 mm.

FIG. 5B is a schematic of the scanning mechanism of the PAM system shown in FIG. 5A. The EOM switches the wavelength from 532 nm to 558 nm after every five lines in the galvanometer (i.e., optical) scanning direction. The blue dots represent A-line acquisitions at 532 nm and the red dots represent acquisitions at 558 nm. In the optical scanning direction, 500 A-lines are acquired over a 4.5 mm range. The mechanical scanning (i.e., B-scan) direction is perpendicular to that of the optical scanning.

FIG. 6A is a graph that represents the optical resolution of the high-speed wide-field multi-parametric PAM and acoustic focal zone of the cylindrical transducer. The optical resolution is quantified by imaging a sharp edge in the USAF resolution target. The blue dots represent the experimental data, and the red line shows the fitted edge-spread function. The blue line shows the derived line-spread function.

FIG. 6B is an image of the raster line from the PAM system. The acoustic focal zone is mapped by a 2-D raster scan of a piece of black tape.

FIG. 6C is a graph of the long axis of the acoustic focal zone, which has an FWHM value of 4.5 mm.

FIG. 6D is a graph of the short axis of the acoustic focal zone, which has an FWHM value of 76 μm.

FIG. 7A is a high-speed wide-field multi-parametric PAM of sO₂ over a 4.5×1 mm² area in the mouse ear at a 1-Hz frame rate. The sO₂ images were acquired under normoxia (left) and two minutes into hypoxia (right).

FIG. 7B is a high-speed wide-field multi-parametric PAM of blood flow speed over a 4.5×1 mm² area in the mouse ear at a 1-Hz frame rate. The flow images were acquired under normoxia (left) and two minutes into hypoxia (right).

FIG. 7C is a graph of the dynamic responses of the average sO₂ and blood flow to the hypoxic challenge. The solid blue curve and red curve show the changes in arterial and venous sO₂, respectively. The dotted blue curve shows the change in blood flow.

FIG. 8A is a high-speed wide-field multi-parametric PAM of sO₂ only over a 4.5×4.5 mm² area in the mouse ear at a 1.3-Hz frame rate. The sO₂ image was acquired under normoxia.

FIG. 8B is a high-speed wide-field multi-parametric PAM of sO₂ only over a 4.5×4.5 mm² area in the mouse ear at a 1.3-Hz frame rate. The sO₂ image was acquired 88 seconds into hypoxia.

FIG. 9 is a flow diagram of two-step sparse coding-based denoising.

FIG. 10 is a schematic of the multi-parametric PAM. AOM: acousto-optic modulator, PM-SMF: polarization-maintaining single-mode fiber; BPF: bandpass filter; OM: dichroic mirror; EOM: electro-optic modulator, PBS: polarizing beamsplitter, BB: beam block, BS: beam sampler, PD: photodiode, OL: objective lens, CL: correction lens, UT: ultrasound transducer, WT: water tank.

FIG. 11A is a step-by-step illustration of the performance of two-step denoising on carbon fiber images acquired by low-fluence PAM (20% of normal fluence). First row: low-fluence (i.e., 1 nJ pulse energy) PAM images of randomly distributed carbon fibers before denoising, after B-scan denoising alone, MAP denoising alone, and two-step denoising, as well as the reference image acquired with normal fluence (i.e., 5 nJ). Second row: Close-up views of non-fiber background.

FIG. 11B is an illustration of the denoising performance in images acquired with 10% and 5% of normal fluence (i.e., 0.5 nJ and 0.25 nJ, respectively) through a side-by-side comparison of the low-fluence images before and after two-step denoising. PA: photoacoustic.

FIG. 12A is a graph that illustrates the performance of B-scan denoising on low-fluence PAM of the carbon fiber phantom. The graph shows the effective suppression of random noise in a representative A-line containing the carbon fiber signal.

FIG. 12B is a graph that shows ineffective suppression of noise with signal-like patterns (indicated by the black arrows) in a representative A-line of the non-fiber background. The A-line signal is converted to a bipolar form by subtracting its mean.

FIG. 13A is a step-by-step illustration of the performance of two-step denoising on cerebrovascular structural images acquired by low-fluence PAM in the live mouse. First row: low-fluence (20 nJ pulse energy) images of the cerebral vasculature before denoising (raw), after B-scan denoising alone, MAP denoising alone, and two-step denoising, as well as the reference image acquired with normal fluence (i.e., 100 nJ). Second row: Close-up views of the blue boxed region, showing the improvement of microvascular visualization (indicated by blue arrows). Third row: Close-up views of the green boxed region, showing the suppression of noise fluctuation in non-vessel background. PA: photoacoustic.

FIG. 13B is a set of pseudocolor-coded maps of the SS IM between the low-fluence PAM images (before and after denoising) and the reference image acquired with normal fluence. PA: photoacoustic.

FIG. 14A is a graph that illustrates the performance of B-scan denoising on low-fluence PAM of the mouse cerebral vasculature in vivo, which shows effective suppression of noise (indicated by the red arrow) with an amplitude comparable to that of the microvascular signal (indicated by the green arrow), in a representative A-line.

FIG. 14B is a graph that shows ineffective suppression of noise with signal-like patterns (indicated by the black arrow) in a representative A-line of the non-vessel background. The A-line signal is converted to a bipolar form by subtracting its mean.

FIG. 15A is a set of PAM images that shows that two-step denoising improves the accuracy of C_{Hb}, SO₂, and blood flow measurements at 20% of normal fluence (i.e., 20 nJ pulse energy). Shown are low-fluence C_{Hb} images before and after denoising, as well as the reference image acquired with normal fluence (i.e., 100 nJ). Pseudocolor-coded maps of the SSIM between low-fluence C_{Hb} images (before and after denoising) and the reference image.

FIG. 15B is a set of PAM images that shows low-fluence sO₂ images before and after denoising, as well as the reference image acquired with normal fluence. Pseudocolor-coded maps of the SSIM between low-fluence sO₂ images (before and after denoising) and the reference image.

FIG. 15C is a set of PAM images that shows low-fluence blood flow images before and after denoising, as well as the reference image acquired with normal fluence. Pseudocolor-coded maps of the SSIM between low-fluence blood flow images (before and after denoising) and the reference image.

FIG. 16A is a set of PAM images that illustrates the performance of two-step denoising on cerebrovascular structural and functional measurements in PAM with 10% and 5% of normal fluence (i.e., 10 and 5 nJ pulse energy, respectively). Raw and denoised cerebrovascular structural images were acquired with 10 nJ and 5 nJ pulse energies, and their SSIM maps against the reference image were acquired with normal fluence (i.e., 100 nJ).

FIG. 16B is a set of PAM images that shows raw and denoised C_{Hb} images acquired with 10 nJ and 5 nJ pulse energy, and their SSIM maps against the reference image were acquired with normal fluence.

FIG. 16C is a set of PAM images that shows raw and denoised sO₂ images acquired with 10 nJ and 5 nJ pulse energy, and their SSIM maps against the reference image were acquired with normal fluence.

FIG. 16D is a set of PAM images that shows raw and denoised blood flow images acquired with 10 nJ and 5 nJ pulse energy, and their SSIM maps against the reference image acquired with normal fluence.

FIG. 17 is a table showing a step-by-step analysis of the effects of two-step denoising on SNR, CNR, and SSIM at different fluence levels in a phantom.

FIG. 18 is a table showing a step-by-step analysis of the effects of two-step denoising on SNR, CNR, and SSIM at different fluence levels in vivo.

FIG. 19 is a table of SSIM between cerebrovascular function measured at low fluences (before and after denoising) and normal fluence.

FIG. 20 is a table of the relative errors (against 100 nJ) in quantitative measurements of cerebrovascular function before and after denoising.

FIG. 21A is a system schematic of the configuration of the high-speed wide-field multi-parametric PAM and scanning mechanism. HWP, half-wave plate; EOM, electro-optic modulator; PBS, polarizing beam splitter; PM-SMF, polarization-maintaining single-mode fiber; BPF, band-pass filter; DM, dichroic mirror; PD, photodetector; GM, galvanometer; UT, ultrasound transducer; Objective, f=60 mm.

FIG. 21B is a scanning mechanism schematic of the high-speed wide-field multi-parametric PAM system. The EOM switches the wavelength from 545 nm to 558 nm after every five lines in the galvanometer (i.e., optical) scanning direction. The blue dots represent A-line acquisitions at 545 nm and the red dots represent acquisitions at 558 nm. In the optical scanning direction, 500 A-lines are acquired over a 4.5 mm range. The mechanical scanning (i.e., B-scan) direction is perpendicular to that of the optical scanning.

FIG. 22 is a set of MP-PAM images of wild-type (WT) mouse cerebral cortex through an open-skull window 80 min after topical application of ATN-224, along with graphs quantifying oxygen saturation, CBF, and CMRO₂. An increase in blood oxygenation of the cortical vasculature was observed, indicating decreased oxygen extraction and consumption due to the downregulation of mitochondrial activity. Data were obtained from three mice, each of which was measured once for O₂ saturation, oxygen extraction fraction (OEF), and cerebral metabolic rate of oxygen (CMRO₂). Error bars represent±s.e.m. White arrows: Arteries; yellow arrows: Veins.

FIG. 23 is a schematic of the high-speed PAM system described in Example 8. HWP, half-wave plate; PM-SMF, polarization-maintaining single-mode fiber; BPF, bandpass filter; DM, dichroic mirror; SMF, regular single-mode optical fiber; GM, galvanometer; CL, correction lens; UT, ultrasonic transducer; UR, ultrasonic reflector.

FIG. 24A is an image of the acoustic focal zone within the horizontal plane, delineated by 2D optical scanning of a black tape, and a graph of acoustic focal diameters along the x and y directions, quantified by FWHM values of the Gaussian-fitted cross-sectional profiles.

FIG. 24B is a scanning scheme of the optical-mechanical hybrid scan along the x and y directions.

FIG. 24C is the scheme of high-speed and real-time contour scanning along the x, y, and z directions.

FIG. 25A is a pair of 3D graphs comparing 2D raster scanning and 3D contour scanning on a curved black tape by the high-speed contour-scanning multi-parametric PAM system.

FIG. 25B is a pair of 3D graphs comparing 2D raster scanning and 3D contour scanning on a cluster of carbon fibers on a curved surface by the high-speed contour-scanning multi-parametric PAM system.

FIG. 25C is a set of multi-parametric PAM images of a live mouse brain acquired by high-speed contour scanning.

FIG. 26A is a set of in vivo PAM images of C_{Hb} over time during acute hypervolemic hemodilution. The white arrows highlight a few representative vessels that show noticeable changes in C_{Hb}, sO₂, and flow speed in response to the acute hemodilution.

FIG. 26B is a set of in vivo PAM images of sO₂ over time during acute hypervolemic hemodilution. The white arrows highlight a few representative vessels that show noticeable changes in C_{Hb}, sO₂, and flow speed in response to the acute hemodilution.

FIG. 26C is a set of in vivo PAM images of flow speed over time during acute hypervolemic hemodilution. The white arrows highlight a few representative vessels that show noticeable changes in C_{Hb}, sO₂, and flow speed in response to the acute hemodilution.

FIG. 27A is a graph of acute hypervolemic hemodilution-induced percentage changes in vessel diameter. Two-way repeated-measures ANOVA was used to compare the diameter changes in arteries and veins. One-way repeated-measures ANOVA was used to compare the structural, functional, and metabolic changes over their corresponding baseline values. Statistical significance is marked with an asterisk, where *, **, ***, and **** respectively represent p<0.05, p<0.01, p<0.001, and p<0.0001. Data are presented as mean±SD.

FIG. 27B is a graph of acute hypervolemic hemodilution-induced percentage changes in C_{Hb}. Two-way repeated-measures ANOVA was used to compare the diameter changes in arteries and veins. One-way repeated-measures ANOVA was used to compare the structural, functional, and metabolic changes over their corresponding baseline values. Statistical significance is marked with an asterisk, where *, **, ***, and **** respectively represent p<0.05, p<0.01, p<0.001, and p<0.0001. Data are presented as mean±SD.

FIG. 27C is a graph of acute hypervolemic hemodilution-induced percentage changes in CBF. Two-way repeated-measures ANOVA was used to compare the diameter changes in arteries and veins. One-way repeated-measures ANOVA was used to compare the structural, functional, and metabolic changes over their corresponding baseline values. Statistical significance is marked with an asterisk, where *, **, ***, and **** respectively represent p<p<0.01, p<0.001, and p<0.0001. Data are presented as mean±SD.

FIG. 27D is a graph of acute hypervolemic hemodilution-induced percentage changes in sO₂. Two-way repeated-measures ANOVA was used to compare the diameter changes in arteries and veins. One-way repeated-measures ANOVA was used to compare the structural, functional, and metabolic changes over their corresponding baseline values. Statistical significance is marked with an asterisk, where *, **, ***, and **** respectively represent p<p<0.01, p<0.001, and p<0.0001. Data are presented as mean±SD.

FIG. 27E is a graph of acute hypervolemic hemodilution-induced percentage changes in OEF. Two-way repeated-measures ANOVA was used to compare the diameter changes in arteries and veins. One-way repeated-measures ANOVA was used to compare the structural, functional, and metabolic changes over their corresponding baseline values. Statistical significance is marked with an asterisk, where *, **, ***, and **** respectively represent p<p<0.01, p<0.001, and p<0.0001. Data are presented as mean±SD.

FIG. 27F is a graph of acute hypervolemic hemodilution-induced percentage changes in CMRO₂. Two-way repeated-measures ANOVA was used to compare the diameter changes in arteries and veins. One-way repeated-measures ANOVA was used to compare the structural, functional, and metabolic changes over their corresponding baseline values. Statistical significance is marked with an asterisk, where *, **, ***, and **** respectively represent p<p<0.01, p<0.001, and p<0.0001. Data are presented as mean±SD.

FIG. 28A is a schematic of the configuration of the thin-film optical-acoustic combiner (OAC) described in Example 9.

FIG. 28B is a graph of the reflection spectrum of the OAC.

FIG. 28C is a graph of (c) Left: beam quality of a focused laser beam in the presence or absence of the OAC (left), and images of the focal spot of the laser beam with and without the OAC (right). Scale bar: 25 μm.

FIG. 28D is a graph of pulse-echo signals of the cylindrically focused transducer with and without the OAC.

FIG. 28E is a graph of the normalized frequency spectra of the pulse-echo signals with and without the OAC.

FIG. 29A is a schematic of the high-speed wide-field multi-parametric PAM described in Example 9. HWP, half-wave plate; EOM, electro-optic modulator; PBS, polarizing beam splitter; BS, beam sampler; PM-SMF, polarization-maintaining single-mode fiber; FC, fiber collimator; DM, dichroic mirror; PD, photodetector; GM, galvanometer; UT, ultrasound transducer; OAC, optical-acoustic combiner; AMP, amplifier; Objective, f=50 mm.

FIG. 29B is the schematic of the hybrid scanning mechanism of the PAM system described in Example 9. The EOM switches the wavelength from 532 nm to 558 nm after every 36 lines in the GM (i.e., optical) scanning direction. The green dots represent A-line acquisitions at 532 nm, while the yellow dots represent acquisitions at 558 nm. In the optical scanning direction, 83 A-lines are acquired in each round trip of the GM scanner. The mechanical scanning (i.e., B-scan) direction is perpendicular to that of the optical scanning.

FIG. 29C is a graph of lateral resolutions of the PAM with and without the OAC.

FIG. 29D is a graph of axial resolutions of the PAM with and without the OAC.

FIG. 30A is a PAM image of sO₂ under normoxia using high-speed multi-parametric PAM over a 4.5×3 mm² area in the awake mouse brain at a frame rate of 0.3 Hz.

FIG. 30B is a PAM image of sO₂ 100 s into hypoxia using high-speed multi-parametric PAM over a 4.5×3 mm² area in the awake mouse brain at a frame rate of 0.3 Hz.

FIG. 30C is a PAM image of blood flow under normoxia using high-speed multi-parametric PAM of over a 4.5×3 mm² area in the awake mouse brain at a frame rate of 0.3 Hz.

FIG. 30D is a PAM image of blood flow 100 s into hypoxia using high-speed multi-parametric PAM of over a 4.5×3 mm² area in the awake mouse brain at a frame rate of 0.3 Hz.

FIG. 30E is a graph of dynamic responses of the average sO₂ and blood flow to the hypoxic challenge. The red curve with dots and the blue curve with triangles show the changes in arterial and venous sO₂, respectively. The black curve with diamonds shows the change in blood flow speed. Scale bar: 1 mm.

FIG. 31A is a high-speed multi-parametric PAM of sO₂ under normoxia over a 4.5×3 mm² area in the awake mouse brain at a frame rate of 2 Hz. Scale bar=1 mm.

FIG. 31B is a high-speed multi-parametric PAM of sO₂ 30 s into hypoxia over a 4.5×3 mm² area in the awake mouse brain at a frame rate of 2 Hz. Scale bar=1 mm.

FIG. 32 is a table of a Polymer material comparison for manufacturing the optical-acoustic combiner (OAC).

FIG. 33A is a graph of a simulation of acoustic transmission through the acrylic film of OAC without attenuation.

FIG. 33B is a graph of a simulation of acoustic transmission through the acrylic film of OAC with attenuation.

FIG. 34 is a photo of the scanning head of the PAM system reported in Example 9. UT, ultrasound transducer; OAC, optical-acoustic combiner.

### DETAILED DESCRIPTION OF THE INVENTION

Uniquely capable of imaging blood perfusion, oxygenation, and flow simultaneously at the microscopic level in vivo, multi-parametric photoacoustic microscopy (PAM) has quickly emerged as a powerful tool for studying hemodynamic and oxygen-metabolic changes due to physiological stimulations or pathological processes. However, the low scanning speed poised by the correlation-based blood flow measurement impedes its application in studying rapid microvascular responses.

Photoacoustic microscopy is capable of imaging blood oxygenation with dual-wavelength excitation (e.g., 532 and 558 nm) and spectroscopic analysis. However, high-repetition-rate nanosecond lasers at 558 nm are not commercially available. Thus, the stimulated Raman scattering (SRS) effect in the optical fiber has been used to shift the wavelength of the output of commercial high-repetition-rate nanosecond laser from 532 to 558 nm. To switch the optical wavelength on a single-pulse basis, an electro-optic modulator (EOM) and a half-wave plate are typically used. Although promising, the EOM-based approach suffers from high cost, low modulation speed, and low damage threshold. To address these limitations, we propose an acousto-optic modulator (AOM)-based approach is described. In various aspects, when the AOM is off, all light can go through the 0th order and be coupled into the polarization-maintaining single-mode fiber (PM-SMF) for stimulated Raman scattering-based wavelength conversion. When the AOM is on, most of the light (˜80%) can be diffracted to the 1st order without wavelength conversion. In the meanwhile, the residual light on the 0th order is too weak to trigger the nonlinear SRS effect, and thus all light can be completely blocked by the bandpass filter. This AOM-based wavelength switching module has the following major advantages: low cost (<$2 k), high modulation rate (>1 MHz), and high damage threshold. As an add-on module, it can be readily integrated with commercially available 532-nm nanosecond lasers for dual-wavelength PAM imaging.

The present disclosure implements an ultrahigh-speed, reflection-mode, wide-field, multi-parametric PAM. In some aspects, this invention features a novel optical-acoustic combining module, which is based on a metallic coated polymer film, and a novel laser pulse deployment design, which helps to reduce the optical fluence and thus phototoxicity.

Multi-parametric photoacoustic microscopy (PAM) is uniquely capable of simultaneous, high-resolution mapping of blood hemoglobin concentration, oxygenation, and flow in vivo. However, its speed has been limited by the dense sampling required for blood flow quantification. In some aspects, the present disclosure uses a cylindrically focused transducer to expand the optical scanning range, a metallic-coated polymer film to combine light excitation and ultrasound detection, and a line-by-line wavelength-switching mechanism to reduce the redundant pulses in sO₂ measurement. Together, these innovation features realize the ultrahigh-speed, reflection-mode, wide-field, multi-parametric PAM.

In some aspects, to overcome the speed limitation, an ultra-high-speed multi-parametric PAM system has been developed, which enables the simultaneous acquisition of ˜500 densely sampled B-scans by superposing the rapid laser scanning across the line-shaped focus of a cylindrically focused ultrasonic transducer over the conventional mechanical scan of the optical-acoustic dual foci. An optical-acoustic combiner is designed and implemented to accommodate the short working distance of the transducer, enabling convenient confocal alignment of the dual foci in the reflection mode. This system enables continuous monitoring of microvascular hemoglobin concentration, blood oxygenation, and flow over a 4.5×3 mm² area in the awake mouse brain with high spatial and temporal resolution (6.9 μm and 0.3 Hz, respectively).

In some aspects, an optical-mechanical hybrid scan strategy can be adopted to simultaneously acquire multiple B-scans while maintaining the dense sampling for the correlation-based blood flow measurement. In an original demonstration, a spherically focused ultrasonic transducer was used. Although the tight acoustic focus offers high sensitivity, the small acoustic detection area limits the range of laser scanning and thus the number of B-scans that can be simultaneously acquired. To overcome this limitation, the spherically focused transducer can be replaced with a cylindrically focused transducer. However, the short working distance of the cylindrically focused transducer, which is required to achieve tight focus along one dimension for sufficient sensitivity, makes the integration of optical excitation and ultrasonic detection in the reflection mode a challenge. Although multiple different strategies have been developed for the integration of light and ultrasound in PAM, none of them can be readily applied to meet the development goal. For example, an acoustic reflector with a central opening for optical excitation was previously developed as an optical-acoustic combiner (OAC) for PAM, but that design is not compatible with the short working distance of the cylindrically focused transducer. The same difficulty is shared by other types of OACs that transmit light and reflect ultrasound, including an optically transparent acoustic reflector (e.g., glass plate), a dual-prism cube with a thin layer of silicone oil in between, and a single prism with an optical index-matching fluid.

Here, a new, small-footprint OAC and the enabled reflection-mode, ultra-high-speed, multi-parametric PAM system are disclosed. Combining a high-repetition-rate pulsed laser, a high-speed resonant galvanometer, a cylindrically focused transducer, and the new OAC, the new multi-parametric PAM has achieved a 112-fold improvement in imaging speed over a previous system—enabling simultaneous imaging of C_{Hb}, sO₂, and blood flow speed over an extended area of 4.5×3 mm² in 3 s. The acquisition time can be further reduced to 0.5 s if the flow measurement (and thus dense sampling) is not required. The utility of this system has been demonstrated in the awake mouse brain by continuous monitoring of the dynamic changes of cerebrovascular blood oxygenation and flow in response to hypoxic challenges.

In various aspects, a high-speed wide-field multi-parametric PAM system is disclosed which addresses the aforementioned trade-off between imaging speed and sensitivity. Taking advantage of a cylindrically focused transducer (focal zone: 76 μm×4.5 mm), the disclosed multi-parametric PAM system has improved the imaging speed by 112 times over that of the previously reported system with an A-line rate of 300 kHz by extending the optical scanning range from 40 μm to 4.5 mm, enabling high-resolution imaging of the vascular structure, sO₂, and blood flow at 1 frame/second over an area of 4.5×1 mm². The imaging region can be extended to 4.5×4.5 mm² without reducing the frame rate, if flow measurement is not required. The performance of the disclosed system has been demonstrated in the mouse ear, where hypoxia-induced dynamic changes in sO₂ and blood flow were monitored.

In various aspects, an implementation of a multi-parametric PAM system for simultaneous high-resolution imaging of sO₂ and blood flow over an extended region of interest (4.5×1 mm²) with sub-second acquisition time is disclosed. By exploiting high-speed optical scanning across the line-shaped focus of the cylindrical transducer while maintaining the dense A-line sampling along the mechanical scanning direction for the correlation-based flow quantification, this system improves the speed of multi-parametric PAM by 112-fold over the previously reported system with a spherically focused transducer. The feasibility of the system was demonstrated in vivo by monitoring the hemodynamic responses of the mouse ear vasculature to acute hypoxic challenges. The short working distance (6 mm) and large footprint of the cylindrically focused transducer (diameter: 13 mm) make it a challenge to implement the disclosed technique in the reflection mode (i.e., optical excitation and acoustic detection on the same side of the object to be imaged). In some aspects, the present disclosure extends the technique into reflection mode that enables high-speed cortex-wide PAM of hemodynamics and metabolism in the mouse brain. Moreover, in some embodiments, the PAM system can be integrated with previously developed techniques for motion correction and super-resolution imaging to further elevate the impact of this technique.

In one aspect, a system for high-speed, reflection-mode, wide-field, multi-parametric photoacoustic microscopy (PAM) is disclosed herein. The output of a nanosecond pulsed laser can go through an optical switch, which consists of a half-wave plate and an electro-optical modulator (EOM). When a low voltage is applied to the EOM, the polarization remains unchanged, and the beam can go through a polarizing beam splitter (PBS) and a polarization-maintaining single-mode fiber (PM-SMF) for stimulated Raman scattering-based wavelength conversion. With the aid of a band-pass optical filter, the Stokes line at 558 nm can be selected. When a high voltage is applied to the EOM, the polarization state of the beam can be rotated by 90 degrees. As a result, the beam is reflected by the PBS. Then, the reflected 532-nm beam and the 558-nm Stokes beam can be combined through a dichroic mirror and coupled into a single-mode optical fiber. Before fiber coupling, a beam sampler can be used to tap off ˜5% of the pulse energy for optical fluence monitoring and compensation by a photodetector. In the imaging head, the dual-wavelength beam can be collimated and then steered by a one-axis galvanometer. To achieve capillary-level lateral resolution, the beam can be focused by an objective lens, which is aligned coaxially and confocally with the cylindrically focused transducer. Reflecting light while transmitting ultrasound, a metallic-coated polymer film can be used to combine the optical and acoustic paths. Thus, the generated ultrasound can pass through the polymer film and can be detected by the cylindrically focused transducer. The galvanometer's voltage can be adjusted to overlap the optical scanning range with the long axis of the ultrasonic focus, and the mechanical scanning direction can be aligned with the short axis of the focus.

In previously reported multi-parametric PAM systems, sO₂ measurements were performed by switching pulse by pulse between 532 and 558 nm. However, the dense sampling required for the flow quantification is overkill for the sO₂ measurement. Thus, instead of switching the wavelength on an individual pulse basis, the laser wavelength can be switched to 558 nm after every five lines of optical scanning at 532 nm in the disclosed system. This laser pulse deployment design helps to reduce the optical fluence and associated phototoxicity.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### Examples

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice.

However, those of skill in the art should, in light of the present disclosure, appreciate that many changes can be made in the specific embodiments that are disclosed and still obtain a like or similar result without departing from the spirit and scope of the present disclosure.

### Example 1—Simultaneous Imaging of Amyloid Deposition and Cerebrovascular Function Using Dual-Contrast Photoacoustic Microscopy

This Example describes simultaneous imaging of amyloid deposition and cerebrovascular function using dual-contrast photoacoustic microscopy.

### Abstract

Pathological aggregation of A/3 peptides results in the deposition of amyloid in the brain parenchyma (senile plaques in Alzheimer's disease [AD]) and around cerebral microvessels (cerebral amyloid angiopathy [CAA]). The current understanding of amyloid-induced microvascular changes has been limited to the structure and hemodynamics, leaving the oxygen-metabolic aspect unattended. In this Letter, a dual-contrast photoacoustic microscopy (PAM) technique is reported, which integrates the molecular contrast of dichroism PAM and the physiological contrast of multi-parametric PAM for simultaneous, intravital imaging of amyloid deposition and cerebrovascular function in a mouse model that develops AD and CAA. This technique opens up new opportunities to study the spatiotemporal interplay between amyloid deposition and vascular-metabolic dysfunction in AD and CAA.

### Methods and Results

Pathological aggregation of misfolded Af3 peptides in the brain has long been associated with both neurodegeneration and cerebrovascular dysfunction. Specifically, the formation of amyloid plaques in the extracellular spaces between neurons is thought to play a key role in Alzheimer's disease (AD), the most common cause of dementia. The deposition of amyloid in the walls of small cortical arterioles and leptomeningeal vessels can lead to cerebral amyloid angiopathy (CAA), a common form of cerebral small-vessel disease and a common feature of AD. Notable evidence has shown that in CAA, amyloid deposition is associated with physiopathological changes in the cerebral microvasculature, including blood flow, reactivity, and oxygen delivery. Thus, understanding the spatiotemporal interplay between amyloid formation and cerebrovascular function in animal models of AD and CAA might provide new insights into the disease mechanisms.

To date, simultaneous amyloid and cerebrovascular imaging in AD and CAA has been primarily carried out using confocal or two-photon microscopy. Most of the studies have been limited to amyloid deposition-induced changes in the microvascular structure and hemodynamics, leaving the oxygen-metabolic aspect unattended. In addition to fluorescence microscopy, optical coherence tomography (OCT) has been applied for amyloid imaging. However, the speckle in OCT limits its specificity and contrast. Besides, stimulated Raman scattering (SRS) microscopy has been recently applied to image amyloid plaques. However, the in vivo application has been impeded by the limited imaging speed and tissue penetration.

Capitalizing on the Brownian motion of red blood cells, the distinct optical absorption spectra of oxy-hemoglobin and deoxy-hemoglobin, and the blood flow-induced signal decorrelation, multi-parametric photoacoustic microscopy (PAM) has been developed for comprehensive imaging of microvascular function, including the total concentration of hemoglobin (C_{Hb}), oxygen saturation of hemoglobin (sO₂), and blood flow speed in the live mouse brain. In parallel, exploiting the unique dichroism contrast of Congo red (CR)-labeled amyloid plaques, the feasibility of using PAM for high-sensitivity, high-specificity imaging of amyloid plaques in histological sections of the AD mouse brain has been demonstrated. When labeled with CR, amyloid plaques present linear dichroism because of the fibril orientation. Specifically, if the fibril orientation is parallel to the polarization state of the light, the optical absorption (and thus the photoacoustic amplitude) reaches the maximum. In contrast, if the fibril orientation is perpendicular to the light polarization, the photoacoustic signal is diminished. The polarization-dependent optical absorption (i.e., dichroism) enables differential detection with orthogonally polarized photoacoustic excitations to achieve background-free amyloid imaging. Together, these studies show the significant potential of PAM for simultaneous imaging of amyloid deposition and cerebrovascular function in AD and CAA in vivo.

In this example, a dual-contrast PAM system is reported that integrates the molecular contrast of dichroism PAM and physiological contrast of multi-parametric PAM for simultaneous, intravital imaging of amyloid deposition and cerebrovascular function in a mouse model developing both AD and CAA. The extension of PAM-based amyloid imaging from plaques, which are spatially separated from the vasculature, to amyloid deposits in the vessel wall has not been demonstrated before. Thus, a two-step validation of the new technique against standard fluorescence microscopy was performed, first in a brain section that contains only CR-labeled amyloid plaques and then in a freshly dissected mouse brain containing CR-labeled amyloid plaques, blood-perfused microvessels, and amyloid deposition in the vessel wall. Upon successful validation, the performance of the dual-contrast PAM was tested for simultaneous imaging of amyloid (in the forms of both senile plaques and deposits in the vessel wall) and cerebrovascular function (including C_{Hb}, sO₂, and blood flow) in the live mouse brain.

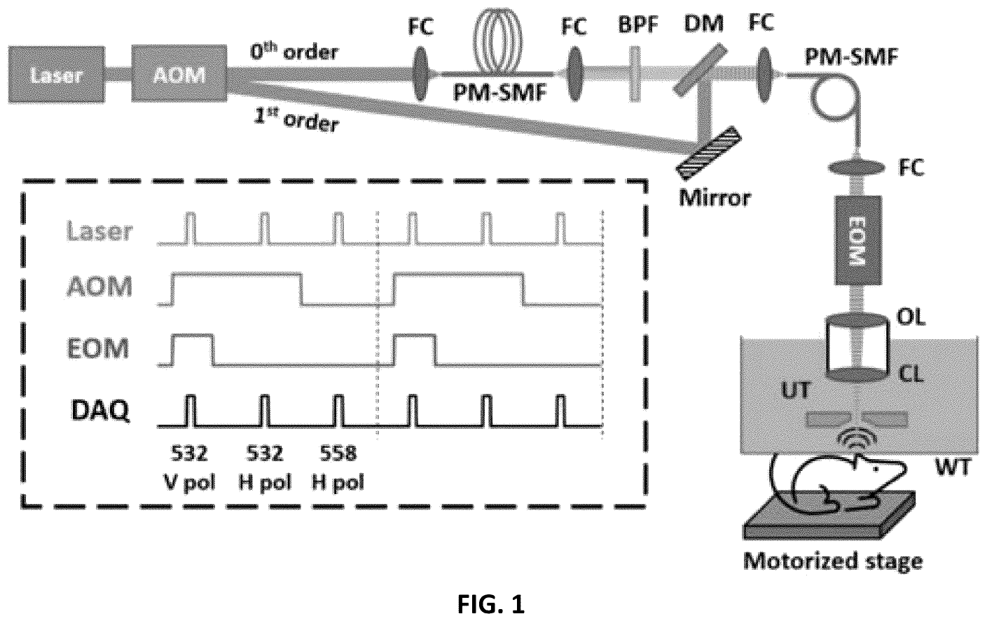

As shown in FIG. 1, the dual-contrast PAM uses a nanosecond-pulsed laser for light excitation (GLPM-10-Y13, IPG Photonics; wavelength, 532 nm; pulse repetition rate used in this Letter, 30 kHz). Individual pulses coming out of this laser are switched between two optical paths by an acousto-optic modulator (AOM; AOMO 3080-122, Crystal Technology). The pulse energy after the AO M is ˜1000 n J. When the AOM is off, the laser light passes it without diffraction (i.e., 0th order) and is coupled into a polarization-maintaining single-mode optical fiber (PM-SMF, HB450-SC, Fibercore) through a fiber coupler (CFC-I IX-A, Thorlabs). The SRS in the PM-SMF redshifts the laser wavelength from 532 to 558 nm. Then a bandpass filter (CT560/10 bp, Chroma) is used to isolate the 558 nm component. Under this condition, the output from the PM-SMF is ˜520 nJ, which is reduced to ˜340 nJ after bandpass filtering. When the AOM is on, ˜60% of the 532 nm light will be diffracted (i.e., 1st order) into the second optical path, where no wavelength conversion is implemented. The remaining 40% of undiffracted light goes through the SRS path. Based on experimental tests using both a power meter and a high-speed photodetector, the threshold for SRS-based generation of 558 nm Stokes light is ˜50% of the input power. Thus, no 558 nm light is generated with 40% of the power, when the AOM is on. The two optical paths are merged by using a dichroic mirror (OM; FF538-FDiO 1, Semrock), and the dual-wavelength beam is coupled into a PM-SMF to maintain the linear polarization.

As shown in the boxed inset of FIG. 1, by triggering the AOM at 10 kHz, the pulse train emitted by the laser operating at a 30 kHz pulse repetition rate is packaged into multiple three-pulse packets, each of which consists of two 532 nm pulses and one 558 nm pulse. An electro-optical modulator (EOM; Model 350-80, Conoptics) is utilized to modulate the polarization states of the two 532 nm pulses to be orthogonal to each other, with a polarization extinction ratio of more than 100:1. The pulsed laser beam, containing two wavelengths (532 and 558 nm) and two polarization states at 532 nm, is focused into the object to be imaged by an objective lens, through a correction lens and then the central opening of a ring-shaped ultrasonic transducer (UT; inner diameter, 1.1 mm; outer diameter, 3.0 mm; focal length, 4.4 mm; center frequency, 40 MHz; 6 dB bandwidth, 69%). For acoustic coupling, the UT and correction lens (CL) are immersed in a homemade water tank, and a thin layer of ultrasound gel is applied between the object and a piece of polyethylene membrane at the bottom of the water tank. Light-excited acoustic waves are detected by the UT, amplified by a low-noise amplifier, and acquired by a high-speed data acquisition board (DAQ, ATS9350, AlazarTech). The object is raster-scanned using two motorized stages to form images. A field-programmable gate array (FPGA, PCie-7842r, National Instruments) is programmed to synchronize the laser, AOMs, EOM, stages, and DAQ for simultaneous acquisition of the dual-contrast images. In this experimental setting, the dwell time of each pixel is ˜100 μs, and the pixel size is ˜0 20 μm×1.67 μm. One set of dual-contrast images can be acquired within min. The spatial resolution of the dual-contrast PAM system is quantified to be ˜31 μm, and the imaging depth is ˜300 μm.

With the specifically designed laser excitation scheme, three images can be simultaneously acquired using a single raster scan, including two images with the orthogonally polarized (i.e., vertical and horizontal) 532 nm light and one image with the horizontally polarized 558 nm light. Subtraction of the two images acquired at 532 nm reveals the dichroism contrast of CR-labeled amyloid deposits, while spectroscopic, statistical, and correlation analyses of the images acquired with the horizontally polarized 532 and 558 nm light provide multi-parametric quantification of microvascular C_{Hb}, sO₂, and blood flow speed. Given the 30 kHz pulse repetition rate and 2 mm/s motor speed, the spatial interval between the two A-lines acquired with adjacent pulses is ˜66.7 nm, which is much smaller than the average sizes of amyloid plaques (>10 μm) and red blood cells (>5 μm). Thus, the mismatch between the three simultaneously acquired images is negligible.

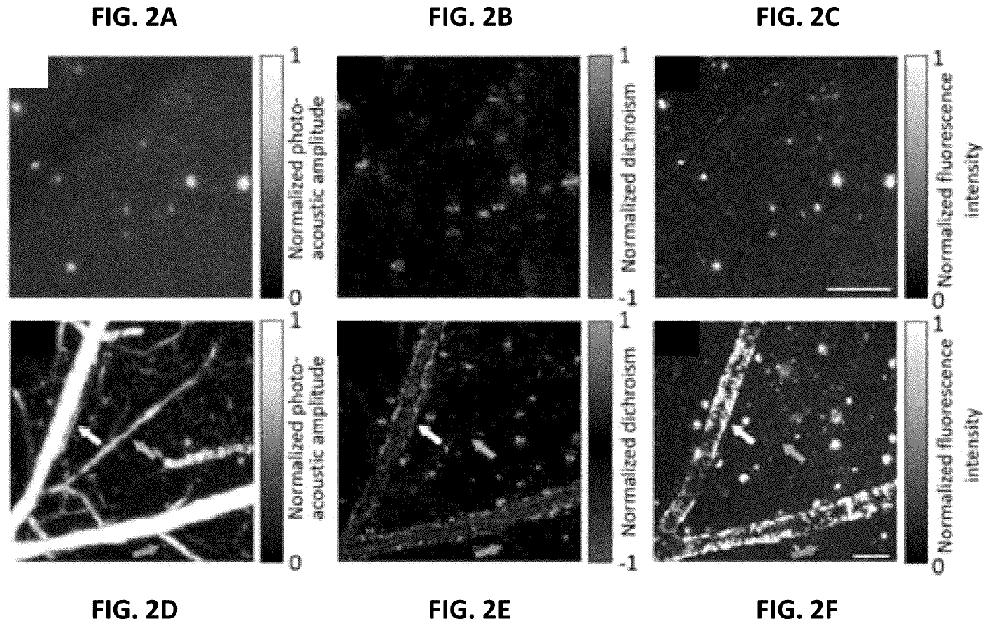

A two-step validation of the dual-contrast PAM was carried out. In the first step, the brain section of a nine-month-old (APP)/PS 1 mouse (Strain No: 34829, Jackson Laboratory) was incubated in a CR solution (2 μg/ml CR in 0.1 M phosphate-buffered saline [PBS], Sigma) for 10 min to label amyloid plaques, rinsed with 0.1 M PBS, and then examined by the dual-contrast PAM. The results show that conventional, amplitude-based PAM presents considerable non-amyloid background due to the limited specificity, as shown in FIG. 2A, acquired with the horizontally polarized 532 nm light. (Note that the amplitude-based image acquired with the vertically-polarized 532 nm light shows a similar background.) In contrast, subtraction of the two amplitude-based images acquired with orthogonally polarized light yields an amyloid-specific dichroism image. As shown in FIG. 2B, the normalized dichroism revealed by the differential detection effectively removes the background and enhances the contrast of amyloid plaques, where the green and red colors, respectively, indicate that PAM signals are predominantly generated by the absorption of horizontally and vertically polarized light. A side-by-side comparison of the plaque image acquired using dichroism PAM with that by a confocal microscope (BX6IWI, Olympus; excitation, 515 nm; detection, 560-660 nm) demonstrates the high specificity of dichroism-based amyloid plaque detection (FIG. 2C).

In the second step, a CR-stained, freshly dissected APP/PS1 mouse brain was imaged to further investigate whether the dual-contrast PAM can image amyloid deposits in the vessel wall, which was more challenging due to the presence of strong photoacoustic signals of the hemoglobin. Following anesthesia with ˜1.0% vaporized isoflurane, the mouse was placed in a stereotactic holder. A midline incision was made, and the periosteum was removed from the cranium. After identification of the Bregma, appropriate coordinates (1 mm lateral and 0.5 mm posterior) were registered for the stereotactic injection. Then a 0.5 mm burr hole was created using a dental drill, and a microsyringe was directed to the recorded coordinates for injection of 5 μL CR. After removing the syringe, the surgical incision was closed with a 4-0 nylon suture. The mouse was returned to its home cage after recovering from anesthesia in a temperature-controlled incubator. Two days after the CR injection, the animal was euthanized, and the brain was dissected and examined by the dual-contrast PAM. The results show that the amyloid deposits in the vessel wall, which are completely indistinguishable from the blood in the amplitude-based PAM images, can be clearly identified using the dichroism contrast and well agree with those observed by the confocal microscope (white and cyan arrows in FIG. 2D-F). Moreover, a plaque-like structure in the amplitude-based PAM image (green arrow in FIG. 2D) is shown not to be an amyloid plaque in the dichroism image (FIG. 2E) and the fluorescence image (FIG. 2F), once again demonstrating the high specificity of the dichroism contrast for amyloid imaging.

To examine the utility of the dual-contrast PAM for simultaneous imaging of amyloid and cerebrovascular function in vivo, the parietal cortex of a 10-month-old APP/PS1 mouse was imaged through a 3×3 mm² cranial window two days after the CR injection. The pulse energy at the tissue surface was kept at about 100 nJ. All experimental procedures were carried out in conformity with the animal protocol approved by the Institutional Animal Care and Use Committee at Washington University in St. Louis.

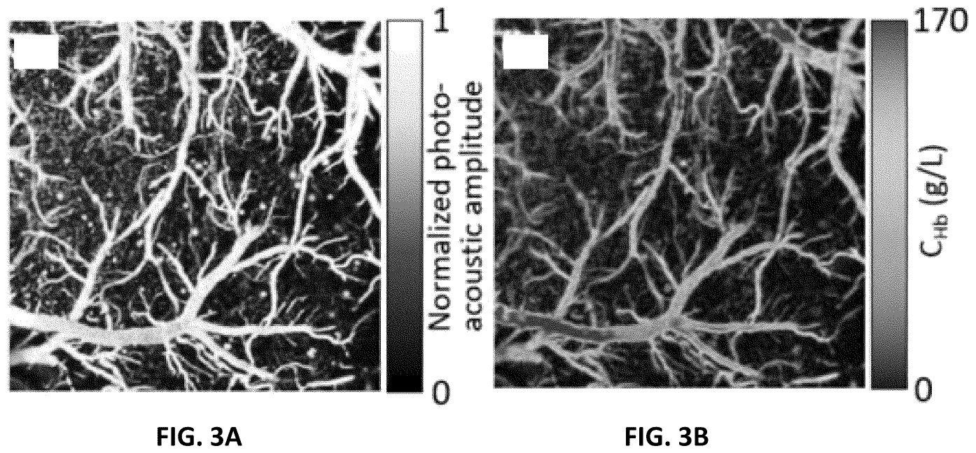

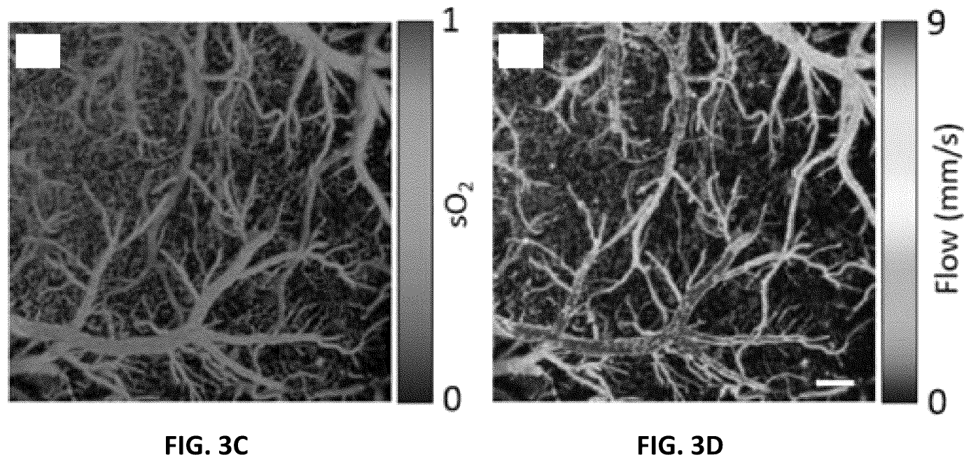

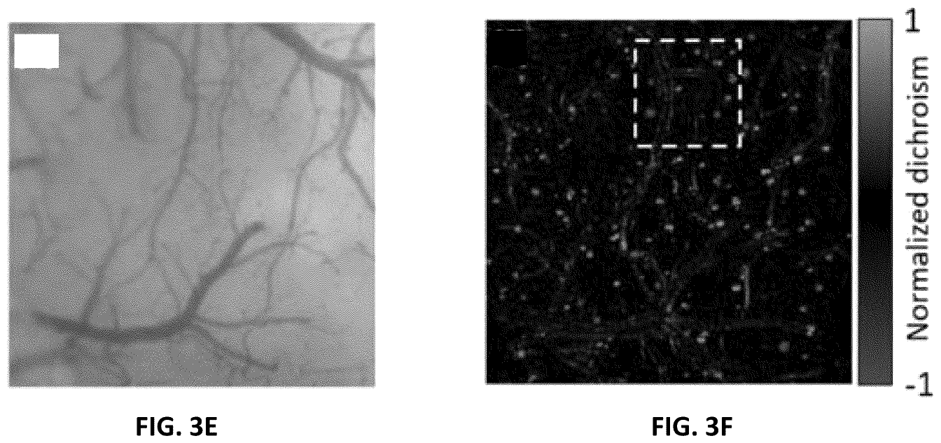

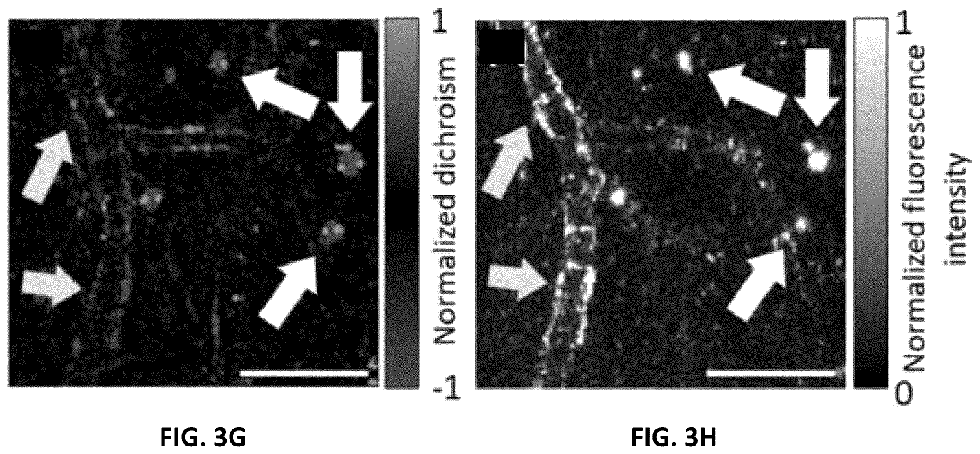

As shown in FIG. 3A-D, the dual-contrast PAM measures the cerebrovascular structure, C_{Hb}, sO₂, and blood flow speed over the 2×2 mm² region of interest (ROI), providing additional microvascular and functional insights in comparison to the picture taken by a wide-field microscope (SM-3TZ-54S, AmScope) over the same ROI (FIG. 3E). The simultaneously acquired dichroism image shows amyloid deposition across the entire ROI (FIG. 3F). A side-by-side comparison of the boxed region imaged by dichroism PAM (FIG. 3G) and confocal microscope (FIG. 3H) shows similar distributions of both amyloid plaques (white arrows) and amyloid deposits in the vessel wall (yellow arrows).

In summary, a dual-contrast PAM technique was developed for simultaneous, intravital imaging of cerebral microvascular physiology and amyloid dichroism. This technique opens up new opportunities to study the spatiotemporal interplay between amyloid and vascular-metabolic dysfunction in AD and CAA.

### Example 2—Laser Wavelength Switching Module for Photoacoustic Microscopy

This Example describes a laser wavelength switching module for photoacoustic microscopy.

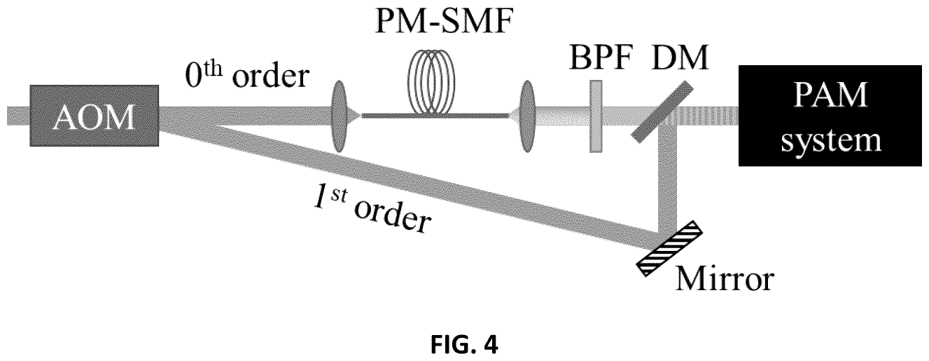

As shown in FIG. 4, when the AOM is off, all light will go through the 0th order and be coupled into the PM-SMF for the SRS effect. When the AOM is on, most of the light (˜80%) will be diffracted to the 1^{st} order without wavelength conversion. In the meanwhile, the residual light on the 0^{th} order is too weak to excite the SRS effect so all light will be completely blocked by the BPF.

### Example 3—Ultrahigh-Speed, Reflection-Mode, Wide-Field, Multi-Parametric Pam

This Example describes ultrahigh-speed, reflection mode, wide-field, multi-parametric PAM.

Uniquely capable of imaging blood perfusion, oxygenation and flow simultaneously at the microscopic level in vivo, multi-parametric photoacoustic microscopy (PAM) has quickly emerged as a powerful tool for studying hemodynamic and oxygen-metabolic changes due to physiological stimulations or pathological processes. However, the low scanning speed poised by the correlation-based blood flow measurement impedes its application in studying rapid microvascular responses. To address this challenge, an implementation of ultrahigh-speed, reflection-mode, wide-field, multi-parametric PAM has been invented.

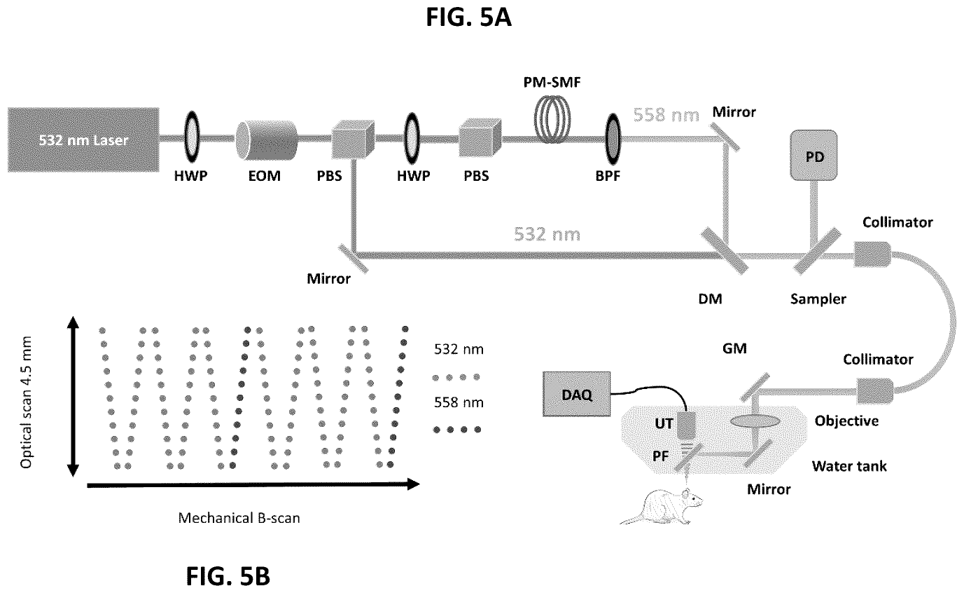

The system schematic is shown in FIG. 5A. The output of a nanosecond pulsed laser goes through an optical switch, which consists of a half-wave plate and an electro-optical modulator (EOM). When a low voltage is applied to the EOM, the polarization remains unchanged, and the beam goes through a polarizing beam splitter (PBS) and a polarization-maintaining single-mode fiber (PM-SMF) for stimulated Raman scattering-based wavelength conversion. With the aid of a band-pass optical filter, the Stokes line at 558 nm can be selected. When a high voltage is applied to the EOM, the polarization state of the beam is rotated by 90 degrees. As a result, the beam is reflected by the PBS. Then, the reflected 532-nm beam and the 558-nm Stokes beam are combined through a dichroic mirror and coupled into a single-mode optical fiber. Before fiber coupling, a beam sampler is used to tap off ˜5% of the pulse energy for optical fluence monitoring and compensation by a photodetector. In the imaging head, the dual-wavelength beam is collimated and then steered by a one-axis galvanometer. To achieve capillary-level lateral resolution, the beam is focused by an objective lens, which is aligned coaxially and confocally with the cylindrically focused transducer. Reflecting light while transmitting ultrasound, a metallic-coated polymer film is used to combine the optical and acoustic paths. Thus, the generated ultrasound passes through the polymer film and is detected by the cylindrically focused transducer. The galvanometer's voltage is adjusted to overlap the optical scanning range with the long axis of the ultrasonic focus, and the mechanical scanning direction is aligned with the short axis of the focus.

In previously reported multi-parametric PAM systems, sO₂ measurements were performed by switching pulse by pulse between 532 and 558 nm. However, the dense sampling required for the flow quantification is overkill for the sO₂ measurement. Thus, instead of switching the wavelength on an individual pulse basis, the laser wavelength is switched to 558 nm after every five lines of optical scanning at 532 nm, as shown in FIG. 5B. This laser pulse deployment design helps to reduce the optical fluence and thus phototoxicity.

### Example 4—High-Speed, Wide-Field, Multi-Parametric Pam

This Example describes a high-speed, wide-field, multi-parametric PAM.

### Abstract

Capable of imaging blood perfusion, oxygenation, and flow simultaneously at the microscopic level, multi-parametric photoacoustic microscopy (PAM) has quickly emerged as a powerful tool for studying hemodynamic and metabolic changes due to physiological stimulations or pathological processes. However, the low scanning speed poised by the correlation-based blood flow measurement impedes its application in studying rapid microvascular responses. To address this challenge, we have developed a new multi-parametric PAM system has been developed. By extending the optical scanning range with a cylindrically focused ultrasonic transducer (focal zone: 76 μm×4.5 mm) for simultaneous acquisition of 500 B-scans, the new system is 112 times faster than our previous multi-parametric system that uses a spherically focused transducer (focal diameter: 40 μm) and enables high-resolution imaging of blood perfusion, oxygenation and flow over an area of 4.5×1 mm² at a frame rate of 1 Hz. The feasibility of this system has been demonstrated in the living mouse ear. Further development of this system into reflection mode (see Example 3) will enable real-time cortex-wide imaging of hemodynamics and metabolism in the mouse brain.

### Introduction

Photoacoustic microscopy (PAM) is a powerful tool in small-animal research. Relying on the optical absorption contrast of blood hemoglobin, PAM can visualize the vasculature and its changes in response to stimulations or under disease conditions in a label-free manner. Enabling simultaneous imaging of blood perfusion, oxygenation (sO₂), and flow speed at the single-capillary resolution, multi-parametric PAM has shed new light on the hemodynamic and metabolic bases of a wide range of diseases. However, multi-parametric PAM uses correlation-based flow measurement, which requires dense A-line sampling and thus limits the B-scan rate. This limitation impedes its application in studying rapid hemodynamics. Recently, several methods have been developed to increase PAM's scanning speed. Based on water-proofing hexagon-mirror or micro-electro-mechanical systems (MEMS), the B-scan rate of PAM can be significantly increased to 900 Hz. Unfortunately, they are not applicable for correlation-based flow quantification due to the large A-line interval and high B-scan rate. Another method takes advantage of the much more relaxed ultrasonic focus compared to the tight optical focus in PAM. By rapidly steering the optical spot within the ultrasonic focal zone (focal diameter: 40 μm), multiple B-scans can be simultaneously acquired to improve the imaging speed. Although promising, the imaging speed of the optical-mechanical hybrid scan-based multi-parametric PAM system is still limited by the ultrasonic focal zone of the transducer. Relaxing the focus of the spherically focused transducer will allow further improvement of the imaging speed, which however is at the expense of system sensitivity.

In this example, a new implementation of high-speed wide-field multi-parametric PAM is presented, which addresses the aforementioned trade-off between imaging speed and sensitivity. Taking advantage of a cylindrically focused transducer (focal zone: 76 μm×4.5 mm), this new multi-parametric PAM system has improved the imaging speed by 112 times over that of a previously reported system with an A-line rate of 300 kHz by extending the optical scanning range from 40 μm to 4.5 mm, enabling high-resolution imaging of the vascular structure, sO₂, and blood flow at 1 frame/second over an area of 4.5×1 mm². The imaging region can be extended to 4.5×4.5 mm² without reducing the frame rate, if flow measurement is not required. The performance of this new generation system has been demonstrated in the mouse ear, where hypoxia-induced dynamic changes in sO₂ and blood flow were monitored.

### Methods and Results

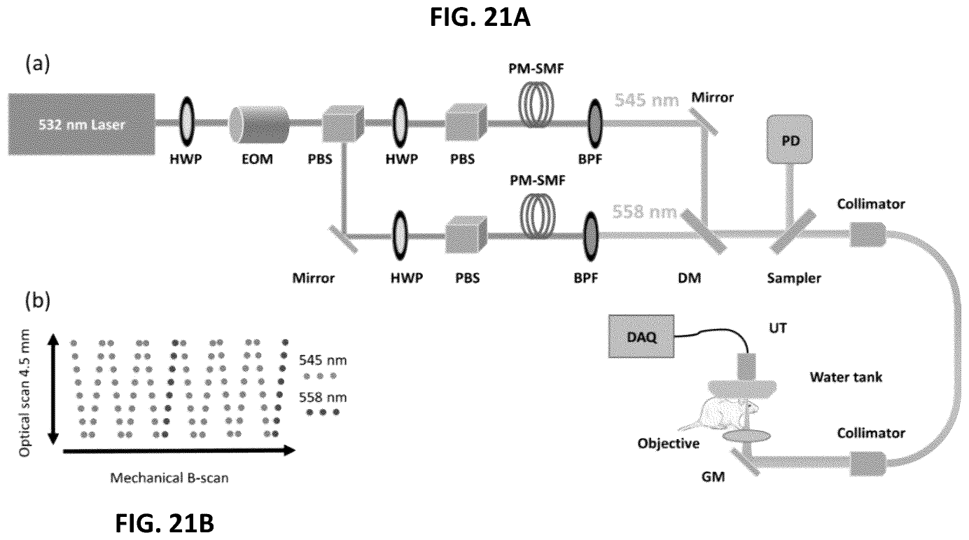

FIG. 21A presents the experimental configuration of the multi-parametric PAM system. The output of a nanosecond pulsed laser (VGEN-G−20, Spectra-Physics; repetition rate: 1 MHz; wavelength: 532 nm) goes through an optical switch, which consists of a half-wave plate (HWP; WPH05M-532, Thorlabs) and an electro-optical modulator (EOM; 350-80, Conoptics). When a high voltage (455 V) is applied to the EOM, the polarization state of the beam is rotated by 90 degrees. As a result, the beam is reflected by a polarizing beam splitter (PBS; PBS121, Thorlabs) and then coupled into a 10-m polarization-maintaining single-mode fiber (PM-SMF; HB450-SC, Fibercore) for wavelength shifting based on the stimulated Raman scattering effect. The output passes through a collimator (CFC-11X-A, Thorlabs) and a band-pass optical filter (BPF; ZET561/10X, Chroma) to isolate the 2nd Stokes line at 558 nm, which is a deoxyhemoglobin absorption-dominant wavelength. When a low voltage (0 V) is applied to the EOM, the polarization remains, and the beam goes through the PBS and a PM-SMF of the same length. By using a different band-pass filter (BPF; ET546/10X, Chroma), the 1st stokes line at 545 nm—an isosbestic point of hemoglobin—is isolated. This process is to minimize the laser leakage at 532 nm, which results in a constant and unnecessary light excitation of the tissue. Since the power of the leakage light is below the threshold of the stimulated Raman scattering, it is not converted to 545 nm and thus is rejected by the band-pass filter. Then, the two Stokes beams are combined using a dichroic mirror (DM; T550lpxr, Chroma) and coupled into a 1 m single-mode fiber (SMF; P1-460B-FC-1, Thorlabs). Before fiber coupling, a beam sampler (BS; BSF10-A, Thorlabs) is used to tap off ˜5% of the pulse energy for optical fluence monitoring and compensation by a photo-detector (PD; PDA25K2, Thorlabs). In the imaging head, the dual-wavelength beam is collimated (Collimator; CFC-11X-A, Thorlabs) and then steered by a one-axis galvanometer (GM; GVS001, Thorlabs) to achieve a scanning range of ±2.5°. To achieve capillary-level lateral resolution, the beam is focused by a plano-convex objective lens (LA1134-A, f=60 mm, Thorlabs), which is aligned confocally with the cylindrically focused transducer (UT; customized by the Ultrasonic Transducer Resource Center at the University of Southern California; Center frequency: 35 MHz). The galvanometer's voltage is adjusted to overlap the optical scanning range with the long axis of the ultrasonic focus, and the mechanical scanning direction is aligned with the short axis of the focus.

The laser beam illuminates the object to be imaged from the bottom. The excited ultrasonic wave is coupled to the transducer immersed in the water tank through a thin layer of ultrasonic gel at the bottom of the water tank and the water inside. A field-programmable gate array (PCIe-7842R, National Instruments) synchronizes the laser, EOM, mechanical-scanning stage, optical-scanning galvanometer, and waveform digitizer board (ATS9350, AlazarTech) for image acquisition.

To measure blood flow at the microscopic level, the correlation-based analysis limits the B-scan speed to 1 mm/s. Using the optical-mechanical hybrid scan, multiple B-scans can be acquired simultaneously to reduce the acquisition time. The utilization of the cylindrical transducer extends the optical scanning range from 40 μm provided by a previously used spherically focused transducer to 4.5 mm, enabling the simultaneous acquisition of 500 B-scans. In the present system, the galvanometer steers the optical focus across the line-shaped ultrasonic focus at 1 kHz. Along the B-scan direction, a step motor moves the object at 1 mm/s for flow quantification. The measurable flow range is co-determined by the pulse repetition rate and B-scan rate. Given a round-trip galvanometer scanning rate of 1 kHz, the maximum measurable flow is 9 mm/s, as experimentally quantified using vessel-mimicking phantoms.

In a previous multi-parametric PAM system, sO₂ measurements were performed by switching pulse by pulse between 532 and 558 nm. By comparing the amplitudes of the photoacoustic signals acquired at the two wavelengths, sO₂ can be quantified. However, the dense sampling required for the flow quantification is overkill for the sO₂ measurement. Thus, instead of switching the wavelength on an individual pulse basis, we switch to 558 nm after every five lines of optical scanning at 545 nm, as shown in FIG. 21B. Along the optical scanning direction, 500 points are acquired in each line. A line of 558-nm pulses is paired with the adjacent line of 545-nm pulses to calculate sO₂. Given the 1-mm/s speed of the mechanical stage and 1-kHz round-trip rate of the galvanometer, the distance between the two pulses at 545 nm and 558 nm used for the sO₂ measurement is <1 μm which is much smaller than the lateral resolution of the PAM system and the diameter of the red blood cell. Thus, the slight spatial mismatch does not affect sO₂ quantification. The distance between two adjacent lines of 558-nm pulses is 3 μm, which is comparable to the average diameter of red blood cells.

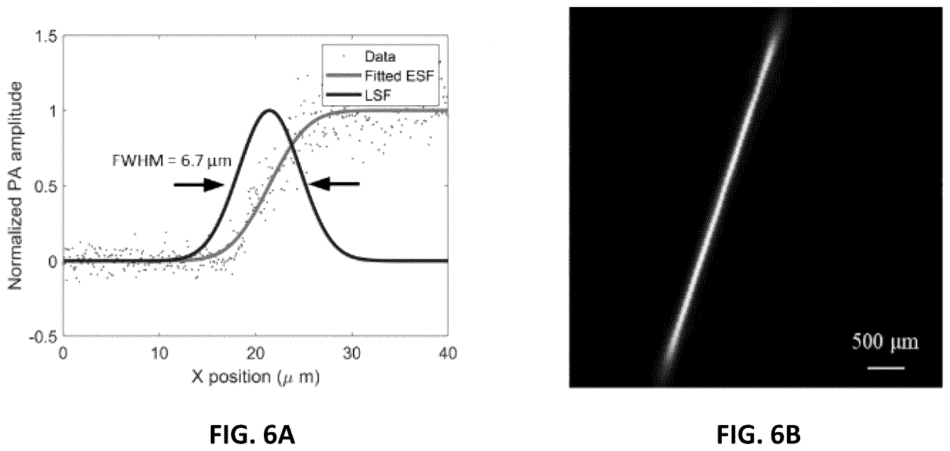

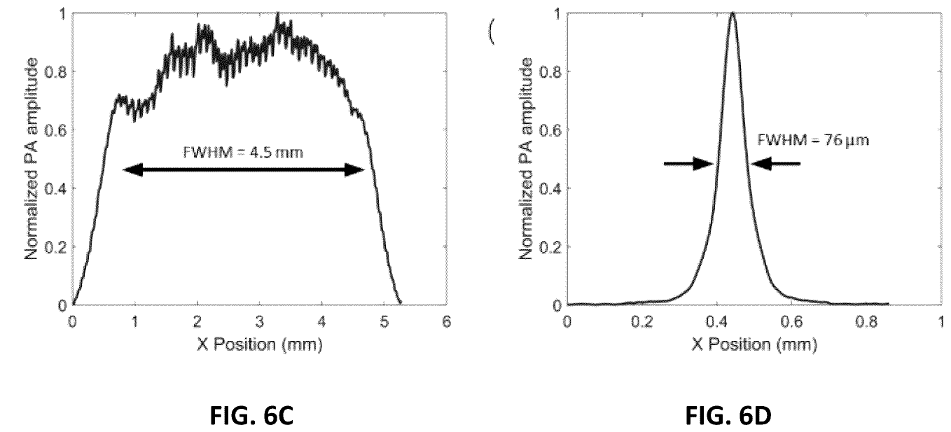

The lateral resolution is quantified by measuring the full-width-at-half-maximum (FWHM) value of the line-spread function (LSF). As shown in FIG. 6A, the edge-spread function is experimentally measured using a U.S. Air Force (USAF) resolution target (R1DS1P, Thorlabs) and then fitted using an error function, from which the LSF is derived and its FWHM value is estimated to be 6.7 μm, in agreement with the theoretical value of 6.1 μm at 545 nm. FIGS. 6B, 6C, and 6D show the quantification of the ultrasonic focus. To characterize the focal zone of the cylindrically focused transducer, the focused light-excitation beam was parked on a piece of black tape to generate a photoacoustic source. Then, the cylindrical transducer is 2-D raster scanned using motorized linear stages to record its response to the light-excited photoacoustic signal, from which the shape of the acoustic focus can be delineated. After mapping the focal zone, the FWHM values along the short and long axes of the ultrasonic focus are estimated to be 76 μm and 4.5 mm, respectively.

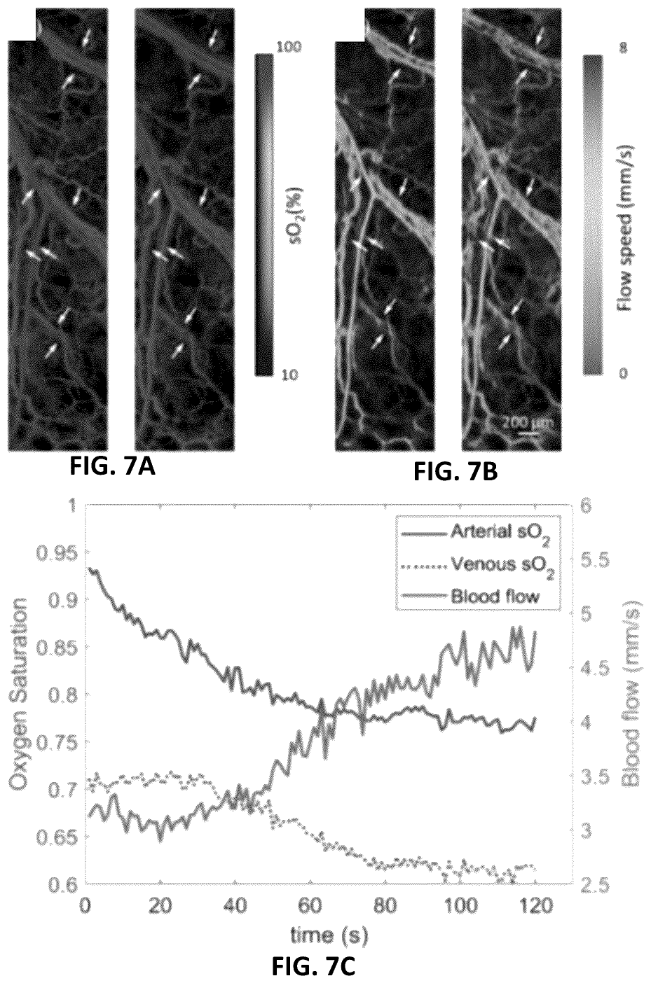

The in vivo performance of this system was tested in the ear of a nude mouse. Throughout the imaging experiment, the mouse was kept under anesthesia with 1.5% isoflurane, and the body temperature was maintained at 37° C. using a heating pad. As shown in the left part of FIGS. 7A and B, the microvascular sO₂ and blood flow speed in the mouse ear were simultaneously imaged over an area of 4.5×1 mm² at a 1-Hz frame rate. In this multi-parametric acquisition mode, the galvanometer scanned across the line-shaped acoustic focus at a round-trip rate of 1 kHz, and the mechanical scanning speed was 1 mm/s. The pulse energy at the surface of the ear was ˜120 nJ.

After the acquisition of baseline images under normoxia, hypoxia was applied by switching the inhalation gas from medical air to hypoxic gas with 10% oxygen concentration. Multi-parametric images were acquired continuously over two minutes. FIGS. 7A and B show the snapshots of the sO₂ and blood flow images acquired before and two minutes after applying hypoxia. The decrease in sO₂ was accompanied by an increase in blood flow in both arteries and veins (highlighted by white arrows). A similar observation has been previously reported in the mouse brain. With the aid of vessel segmentation, the average arterial and venous sO₂ values, as well as the blood flow speed, in the segmented arteries and veins (white arrows in FIGS. 7A and B) over the entire area were quantified. As shown in FIG. 7C, the high-speed hemodynamic recording shows that the arterial sO₂ (blue solid curve) dropped instantly after the onset of hypoxia. In contrast, the venous sO₂ (blue dotted curve) remained unchanged until 20 seconds after applying hypoxia, then started to decrease, and finally plateaued after 90 seconds into hypoxia. The time course of the flow response closely followed that of the venous sO₂, with a correlation coefficient of 0.96.

In cases where blood flow information is not of interest, the imaging area can be extended to 4.5×4.5 mm², while maintaining the frame rate at 1.3 Hz. In this sO₂-only mode, the mechanical-scanning speed is set to 6 mm/s, while the round-trip rate of the optical scanning remains at 1 kHz. Due to the increased step size of the mechanical scanning, the modulation frequency of the EOM was increased to 250 Hz, producing one line of 558 nm pulses after every three lines of optical scanning at 545 nm.

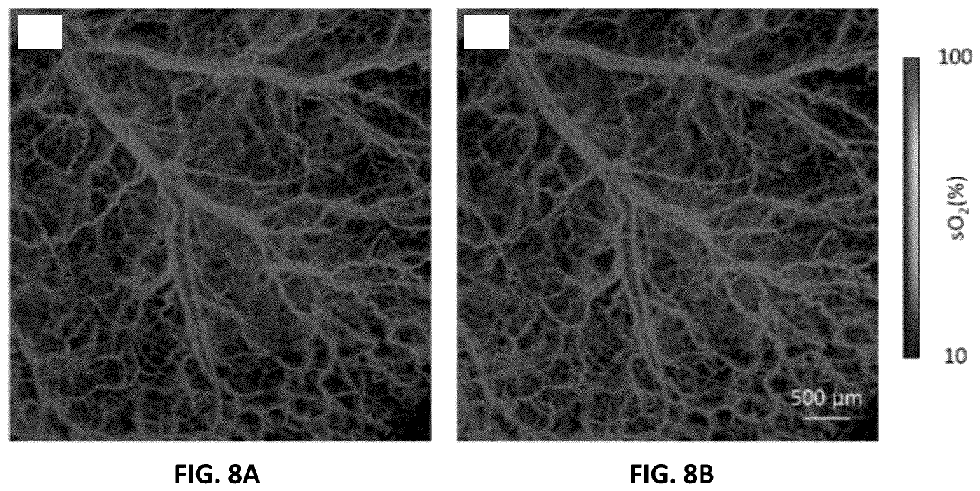

A similar hypoxia experiment was carried out to demonstrate the capability of high-speed wide-field sO₂ monitoring only. FIGS. 8A and B show the sO₂ images acquired under normoxia and 88 seconds into hypoxia, respectively. Dynamic changes in the microvascular sO₂ were recorded at a frame rate of 1.3 Hz.

In conclusion, a new implementation of multi-parametric PAM for simultaneous high-resolution imaging of sO₂ and blood flow over an extended region of interest (4.5×1 mm²) with sub-second acquisition time has been developed. By exploiting high-speed optical scanning across the line-shaped focus of the cylindrical transducer while maintaining the dense A-line sampling along the mechanical scanning direction for the correlation-based flow quantification, this new system improves the speed of multi-parametric PAM by 112-fold over the previously reported system with a spherically focused transducer. The feasibility of the system was demonstrated in vivo by monitoring the hemodynamic responses of the mouse ear vasculature to acute hypoxic challenges. The short working distance (6 mm) and large footprint of the cylindrically focused transducer (diameter: 13 mm) make it a challenge to implement the reported technique in the reflection mode (i.e., optical excitation and acoustic detection on the same side of the object to be imaged). Efforts to address this challenge and extend this technique into reflection mode (See Example 3) enable high-speed cortex-wide PAM of hemodynamics and metabolism in the mouse brain. Moreover, potential integration with previously developed techniques for motion correction and super-resolution imaging may further elevate the impact of this technique.

### Example 5—High-Speed, Wide-Field, Multi-Parametric Pam-Sparse Coding-Enabled Low-Fluence Multi-Parametric Photoacoustic Microscopy

This Example describes sparse coding-enabled low-fluence multi-parametric photoacoustic microscopy.

### Abstract

Uniquely capable of simultaneous imaging of the hemoglobin concentration, blood oxygenation, and flow speed at the microvascular level in vivo, multi-parametric photoacoustic microscopy (PAM) has shown considerable impact in biomedicine. However, the multi-parametric PAM acquisition requires dense sampling and thus a high laser pulse repetition rate (up to MHz), which sets a strict limit on the applicable pulse energy due to safety considerations. A similar limitation is shared by high-speed PAM, which also uses lasers with high pulse repetition rates. To achieve high quantitative accuracy besides good structural visualization at low levels of laser fluence in PAM, a new, sparse coding-based two-step denoising technique has been developed. In the setting of intravital brain imaging, it was demonstrated that this unsupervised learning approach enabled the reduction of the laser fluence in PAM by 5 times without compromising the image quality (structural similarity index measure or SSIM: >0.92) or the quantitative accuracy (errors: <4.9%). Offering a significant relaxation in the requirement of PAM on laser fluence while maintaining the quality of structural imaging and accuracy of quantitative measurements, this sparse coding-based approach is expected to facilitate the application and clinical translation of multi-parametric PAM and high-speed PAM, which have a tight photon budget due to either safety considerations or laser source limitations.

### Introduction

Highly sensitive to optical absorption-based molecular contrast, photoacoustic microscopy (PAM) has attracted considerable attention since being introduced to the biomedical community as an intravital imaging technique. Capitalizing on the light absorption of hemoglobin, PAM enables label-free, comprehensive characterization of microvascular structure and function in vivo. Providing new functional and oxygen-metabolic insights into various physiological and pathological processes, PAM has found broad applications in both basic and translational biomedicine.

Recent advances in the multi-parametric PAM, which enables simultaneous imaging of the hemoglobin concentration (C_{Hb}), oxygen saturation of hemoglobin (sO₂), and blood flow at the microscopic level, further expand its promise. However, quantification of C_{Hb} and blood flow relies on dense sampling, which requires a high laser pulse repetition rate (PRR). Moreover, recent efforts on improving the speed of PAM boost the use of lasers with high PRRs (up to MHz), which leads to increased photon energy deposition in biological tissue per unit time and thus limits the applicable pulse energy due to laser safety considerations. Besides safety concerns, high laser fluence may cause the saturation of optical absorption and thus inaccurate measurement of sO₂.

Although imperative, achieving high structural image quality and quantitative accuracy with low-fluence excitation remains a challenge. At low-fluence levels, the signals generated by the microvasculature are comparable to the noise of PAM systems, resulting in a low signal-to-noise ratio (SNR) that is inadequate for microvascular visualization. Even if some microvessels remain visible under low-fluence excitation, the reduced SNR causes errors in functional measurements as shown by us before. To address this challenge, different techniques have been developed/adopted to improve the quality of low-fluence photoacoustic images, among which sparse coding has shown strong promise for denoising and artifact removal.

Widely used in computer vision and image processing, sparse coding is an unsupervised learning method seeking to represent the image data with a sparse, linear combination of dictionary atoms. Given that unfeatured noise patterns are less correlated and have less sparse representations than signals, sparse coding can differentiate them and has been applied by us and others to remove noise and artifacts in the structural images acquired by PAM. Although demonstrating marked improvement in the SNR of trunk vessels, these efforts have not led to appreciable enhancement in microvascular visualization. More importantly, most of the current studies have been limited to improving the structural image quality-leaving quantitative imaging of the microvascular function unattended. Recently, a deep learning-based technique was developed for denoising the maximum amplitude projection (MAP) image in PAM. Although quantitative imaging of the sO₂ is achieved with a 50% reduction in the laser pulse energy, this method cannot improve the quantification of blood flow, which requires direct analysis of depth-resolved A-lines.

In this example, a new two-step sparse coding-based image processing technique is presented that enables significantly enhanced microvascular visualization and highly accurate quantification of microvascular functions in multi-parametric PAM with low-fluence excitation. After testing the feasibility of this technique in a fiber phantom, its utility is demonstrated by imaging the microvascular structure, C_{Hb}, sO₂, and blood flow speed in the same mouse brain at normal (100 nJ pulse energy) and reduced (20, 10, and 5 nJ) fluence levels. Comparison of the structural and functional images before and after the two-step denoising against those acquired with the normal laser fluence provides a comprehensive assessment of its performance in vivo.

### Methods

### K-SVD-Based Sparse Coding

Under the sparse assumption, the image data admits a sparse decomposition over an overcomplete dictionary. The goal of sparse coding is to describe the data with a trained dictionary and a sparse coefficient matrix. In this example, the K-SVD algorithm is used to train the dictionaries because of its efficiency and simplicity.

Given a noisy image y, the goal is to define an overcomplete dictionary D and identify a sparse coefficient matrix x, which together best represent a noise-free version of the image

ŷ≈Dx. (1)

The process can be described as an optimization problem

$$\\ \begin{matrix}( x , D ) = {arg min}_{x , D} {‖ x ‖}_{0} , s . t . {‖ y - Dx ‖}_{2}^{2} \leq ϵ , & ( 2 )\end{matrix}$$

in which ϵ is related to the noise in the raw image y. Since x is sparse, this problem can be rewritten into

$$\\ \begin{matrix}( x , D ) = {arg min}_{x , D} {‖ y - Dx ‖}_{2}^{2} , s . t . {‖ x ‖}_{0} \leq S , & ( 3 )\end{matrix}$$

where S is the desired sparsity (i.e., the largest number of non-zero entries of x).

When sparse coding is applied to process a large image, the raw image is usually divided into small patches. In this case, the optimization problem can be solved for each catch as

$$\\ \begin{matrix}( {x}_{i} , D ) = {arg min}_{{x}_{i} , D} {‖ {R}_{i} y - {Dx}_{i} ‖}_{2}^{2} , s . t . {‖ {x}_{i} ‖}_{0} \leq S , & ( 4 )\end{matrix}$$

where i denotes the patch index, and operator R; extracts patch i from the original large image y.

To solve this problem, first, the K-SVD algorithm initializes the dictionary and coefficient matrix as D and xᵢ, respectively. Specifically, the dictionary is initialized by a randomly valued matrix and the coefficient matrix is approximated by using the orthogonal matching pursuit (OMP) algorithm. With this, the residual of an arbitrary column c in D can be computed as

eᵢ^{c}=Rᵢy−Σ_{k≠c}d^{k}xᵢ^{k}=Rᵢy−\hat{D}\hat{x}ₗ+d^{k}xᵢ^{c},i∈P_{c}, (5)

where dₖ and x^{k}ᵢ are the k^{th} columns of D and xᵢ, respectively, and P_{c} is the set of patches that use atom dₖ: P_{c}={i|x^{c}ᵢ≠0}. Traversing all columns leads to a residual matrix

E_{c}={eᵢ^{c}},i∈P_{c}, (6)

Then, the dictionary D is updated by minimizing the difference between Rᵢy and Dxᵢ, which can be solved by approximating E_{c} with a rank-one matrix via the singular value decomposition as

E_{c}=UΣV^{T}, (7)

where Σ is the diagonal singular values matrix, and U and V are the left and right singular vectors, respectively. Column dₖ of the updated dictionary is the first column of U. The coefficient x^{c}ᵢ is calculated by multiplying the first column of V by Σ(1,1). After all columns are updated, a new coefficient matrix can be generated using the OMP algorithm. Iterative updates of both the dictionary and the coefficient matrix eventually solve the optimization problem and generate a noise-free image.

### Two-Step Denoising Strategy

Combining two-dimensional (2-D) transverse scan and time-resolved ultrasonic detection, PAM produces three-dimensional (3-D) image sets, consisting of a series of cross-sectional scans (i.e. B-scans) acquired at different tissue locations. Due to the considerable anisotropy in spatial resolution (lateral resolution: a few μm; axial resolution: tens of μm), PAM images are often presented in 2-D by projecting the maximum amplitude of each A-line along the axial/depth direction (i.e. MAP images).

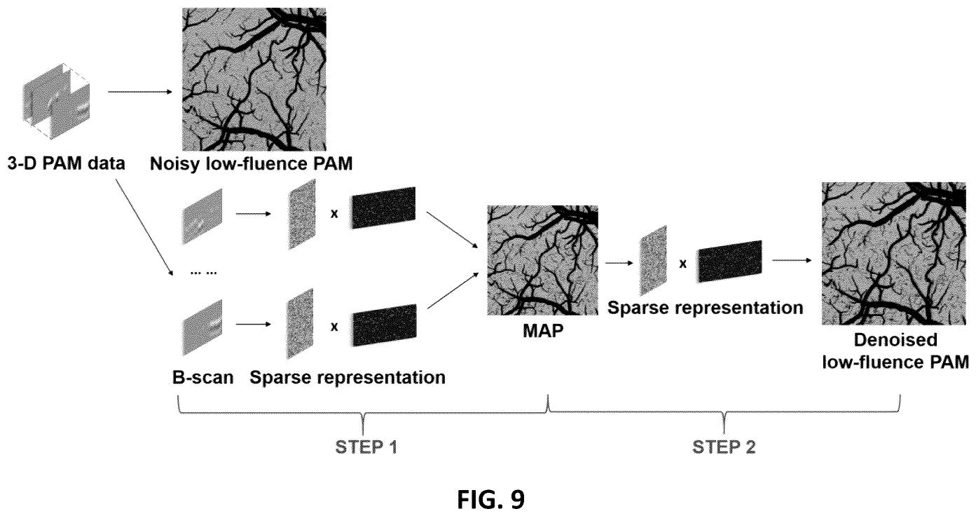

To fully exploit the 3-D imaging nature of PAM, a two-step sparse coding-based denoising strategy is proposed. As shown in FIG. 9, sparse coding is applied first to each raw B-scan acquired by low-fluence PAM and then to the MAP image composited by the denoised B-scans. The two steps are complementary. In the first step, sparse coding can effectively separate the vascular signals from random noise in individual A-lines of the B-scan because the noise is much less sparse compared to the signals. Removing the noise while preserving the weak microvascular signals in A-lines can significantly enhance the visualization of microvessels. However, the noise and possible electromagnetic interference (EMI) that present in patterns similar to that of the spike-like vascular signal in A-lines remain largely unaffected. In the second step, the MAP image, where the vascular pattern is distinct from those of the noise and possible EMI, is sparsely coded by the K-SVD algorithm for separation and removal of the residual non-vessel components. Combining these two steps of denoising results in a near-background-free vascular image. The same K-SVD algorithm is applied in both steps for sparse coding. Note that no other signal processing is involved besides the two-step sparse coding-based denoising.

In this denoising technique, there are four key parameters: dictionary atom size, desired sparsity, patch size, and iteration number. Proper selection of these hyperparameters is essential because of the tradeoff between removing noise and preserving signal. For example, smaller desired sparsity allows better noise suppression, but an excessively small desired sparsity may lead to changes in the amplitude or profile of vascular signals and affect quantitative measurements. Also, a larger atom size better preserves vascular signal for quantitative measurements, but an overly large atom size may compromise the efficacy of noise removal. Moreover, the computational cost is a practical factor to consider when selecting the hyperparameters. Thus, the patch size and iteration number should not be too large while ensuring convergence.

Balancing the denoising performance, quantitative accuracy, and computational cost, we have determined the parameters for the two-step sparse coding-based technique as follows:
Step 1. atom size=50, desired sparsity=3, patch size=100×1, iteration=50.
Step 2. atom size=50, desired sparsity=7, patch size=250×1, iteration=80.

### Experimental Setup

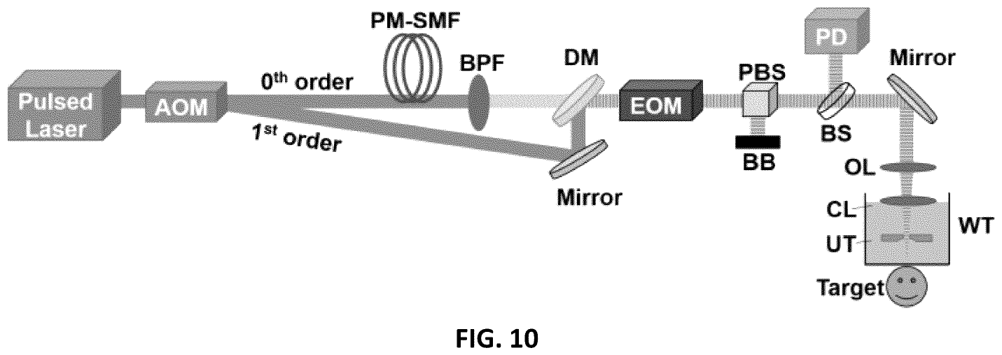

A self-developed multi-parametric PAM system was used in this study. As shown in FIG. 10, the 532-nm output from a nanosecond pulsed laser (GLPM-10, IPG Photonics) is launched into an acousto-optic modulator (AOM, AOMO 3080-122, Crystal Technology) for pulse-by-pulse wavelength conversion. When the AOM is off, the pulsed light undergoes no diffraction and is coupled into a polarization-maintaining single-mode fiber (PM-SMF, HB450-SC, Fibercore), in which the light wavelength is red-shifted due to the stimulated Raman scattering effect. The fiber output then passes a bandpass filter (BPF, CT560/10 bp, Chroma) to select out the 558-nm component. When the AOM is on, 60% of the 532 nm light is diffracted into a different optical path (i.e., 1st-order diffraction), where it experiences no wavelength conversion. The undiffracted (i.e., 0th-order) light, accounting for 40% of the energy, is insufficient to generate nonlinear Raman scattering and thus is removed by the BPF. As a result, the AOM switches the wavelength of the laser pulses between 532 and 558 nm. The two optical paths are combined by a dichroic mirror (DM, FF538-FDiO 1, Semrock). The energy of each laser pulse is modulated by an electro-optic modulator (EOM, 350-80, Conoptics) combined with a polarizing beam-splitter (PBS, PBS121, Thorlabs). To compensate for possible laser fluctuation, ˜5% of the laser light is tapped off by a beam sampler (BS, BSFIO-A, Thorlabs) and monitored by a high-speed photodiode (PD, PDA36A2 Thorlabs). An objective lens (OL, AC254-050-A, Thorlabs) focuses the beam onto the object to be imaged through a ring-shaped ultrasonic transducer (UT, inner diameter: 1.1 mm; outer diameter: 3.0 mm; focal length: 4.4 mm; center frequency: 40 MHz; 6-dB bandwidth: 69%). For acoustic coupling, the transducer is submerged into a water tank (WT) and a thin layer of ultrasound gel (Aquasonic CLEAR, Parker Laboratories) is applied between the target and the tank bottom. A correction lens (CL, LA1207-A, Thorlabs) is used to compensate for the optical aberration induced at the interface of the ambient air and water.

By adjusting the voltage applied to the EOM, the laser pulse energy on the target is altered between 5, 1, 0.5, and 0.25 nJ for phantom imaging and 100, 20, 10, and 5 nJ for in vivo imaging, allowing simultaneous PAM of the same region of interest at different fluence levels. For in vivo experiments, the laser safety standards defined by the American National Standards Institute (ANSI) are considered when determining laser fluence. The highest fluence levels (in the case of 100-nJ laser pulses) are 19.7 mJ/cm2 (532 nm) and 18.0 mJ/cm² (558 nm) at the surface of the mouse brain (beam waist: 1.75 μm, focal depth: 130 μm), which are within the ANSI limit (i.e., 20 mJ/cm²). For phantom imaging, the fluence is set at a much lower level because carbon fibers generate much stronger photoacoustic signals compared to microvessels in the mouse brain. Specifically, we used 5 nJ as normal fluence and 20%, 10%, and 5% of 5 nJ as low fluences (i.e., 1, 0.5, and 0.25 nJ), keeping the same ratio as the in vivo imaging to better benchmark the performance of our denoising method. Structural images of the carbon fibers and cerebral vasculature are generated by the Hilbert transform and maximum amplitude projection of the depth-resolved A-lines, and C_{Hb}, sO₂, and blood flow images of the cerebral vasculature are generated by the statistical, spectroscopic, and correlation analyses, respectively.

The phantom used in this study was randomly placed carbon fibers (average diameter: 6 μm). The in vivo experiment was performed in the brain of a CD-1 mouse (male, 12 weeks old, Charles River Laboratories) through a cranial window. During the imaging experiment, the animal was anesthetized with 1.5% isoflurane, and the body temperature was kept at 37° C. using a temperature-controlled heating pad (Cole-Parmer, EW-89802-52 and Omega, SRFG-303110). All procedures were carried out in conformity with the laboratory animal protocol approved by the Institutional Animal Care and Use Committee (IACUC) at Washington University in St. Louis.

In this example, all data were processed in MATLAB (R20 I 9b, Math Works) using a personal computer (Intel i7-7700 CPU @ 3.60 GHz). For the in vivo dataset, in the first step, 450 B-scan frames (128×7500 pixels each) were processed sequentially at a speed of 24 seconds/B-scan. In the second step, it took 300 seconds to process one MAP image (450×7500 pixels). The total runtime of the denoising algorithm was 3 hours without paralleling computation.

### Quantitative Assessment of Denoising Performance

To quantitively assess the performance of the sparse coding-based two-step denoising technique, multiple key parameters, including the SNR, contrast-to-noise ratio (CNR), and structural similarity index measure (SSIM), are assessed and compared.

The SNR is defined as

SNR=I̅/σₙ, (8)

where I is the average amplitude of the vascular signal, and σₙ is the standard deviation of the amplitude of background noise.

The CNR is defined as

CNR=(Ī−Īₙ)/σₙ (9)

where Iₙ is the average amplitude of the background.

The SSIM, a quantitative measure of the similarity between two images, is defined as

SSIM(x,y)=l^{α}(x,y)c^{β}(x,y)s^{γ}(x,y), (10)

in which l(x,y), c(x,y), and s(x,y) respectively measure the differences between the luminance, contrast, and structure of the two images, and α, β, and γ are three constants. The SSIM map and average SSIM value of the result images are quantified against the reference images acquired with the normal fluence (i.e., 5 nJ or 100 nJ). By selecting the default value of 1 for α, β, and γ, a larger SSIM value indicates higher similarity. Note that since our PAM can simultaneously acquire multiple images at different fluence levels (i.e., 5, 1, 0.5, and 0.25 nJ or 100, 20, 10, and 5 nJ), no image registration is needed prior to the SSIM calculation.

### Results

First, the feasibility of the two-step sparse coding-based denoising technique was demonstrated in a carbon fiber phantom by processing and comparing the images acquired at normal (pulse energy: 5 nJ) and low fluence levels (1, 0.5, and 0.25 nJ).

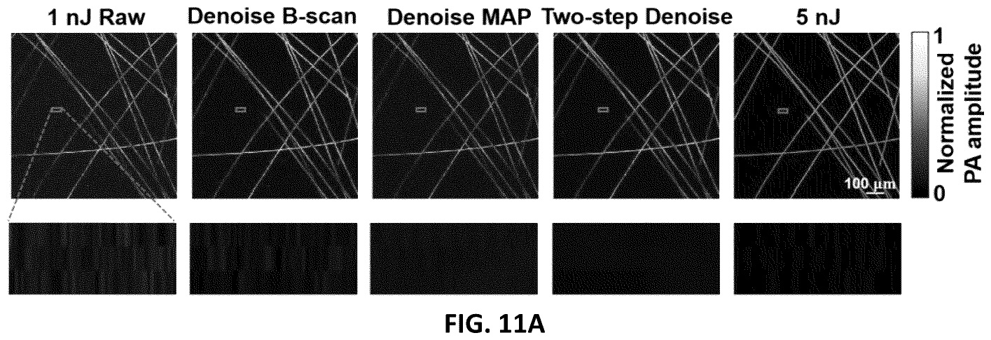

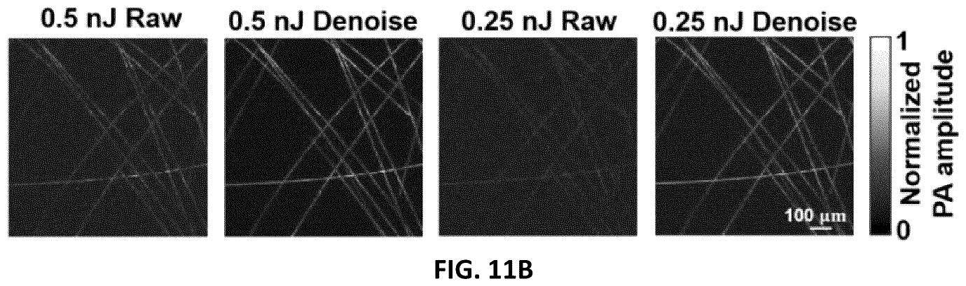

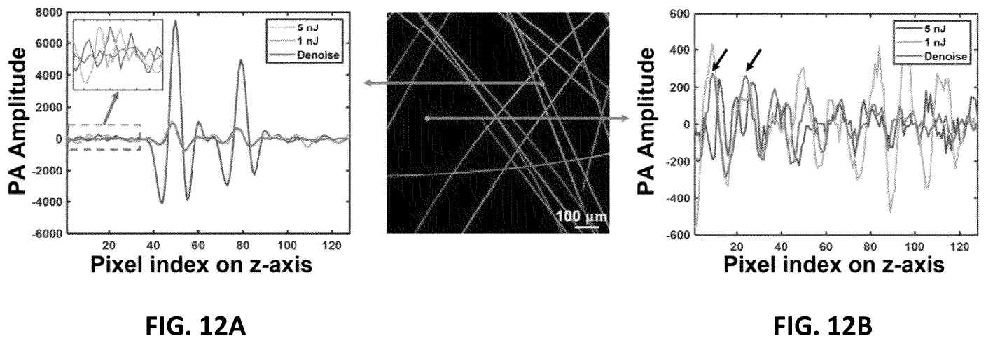

As shown in FIG. 11A, the raw image of the phantom acquired with 20% of the normal fluence (1 nJ) shows considerable noise. Sparse coding-based denoising of the B-scans (i.e., Step 1) improves the visualization of carbon fibers by reducing the noise in individual A-lines (as shown in FIG. 12A). However, the noise and possible EMI that have signal-like patterns in A-lines, remain largely unremoved (indicated by black arrows in FIG. 12B) and present as background fluctuation in the MAP image (the second row of FIG. 11A). By contrast, directly denoising the MAP image with sparse coding (i.e., Step 2) significantly suppresses the background fluctuation. However, the average amplitude of the background remains high, which hinders the enhancement of the image contrast, and thus impedes the improvement of microvascular visibility. Combining the two steps yields the best performance and generates a denoised image, whose quality is comparable to that acquired with the normal fluence (i.e. 5 nJ). Further testing of the denoising technique in carbon fiber images acquired at 10% and 5% of the normal fluence (i.e., 0.5 and 0.25 nJ), which are even noisier and have poor visualization of the fibers, shows that the two-step denoising significantly improves the image quality (FIG. 11B).

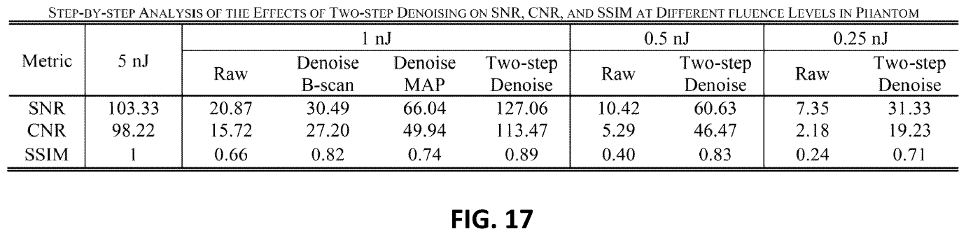

To benchmark the performance of the denoising technique, the key parameters of the raw and denoised images, including the SNR, CNR, and SSIM (against the reference image acquired with 5 nJ pulse energy), are quantified and compared. As shown in FIG. 17, the two-step denoising technique improves the SNR and CNR of the low-fluence images acquired with 20%, 10%, and 5% of the normal fluence by 4.3-6.1 times and 7.2-8.8 times, respectively, whereas the SSIM is increased by 0.23-0.47. The lower the laser fluence, the larger the improvements in the CNR and SSIM. Moreover, step-by-step analysis of the performance of the two-step denoising technique on the image acquired with 20% of the normal fluence shows that denoising the B-scans (Step 1) results in a larger improvement in the SSIM compared to directly denoising the MAP image (Step 2), indicating a better visualization of the carbon fiber structure. By contrast, directly denoising the MAP image leads to a larger improvement of the SNR and CNR, suggesting a more effective suppression of background noise.

Then, the utility of the two-step sparse coding-based denoising for enhancing the microvascular visualization and hemodynamic quantification accuracy of low-fluence PAM was examined in an intravital brain imaging setting. Specifically, the brain of a live CD-1 mouse was concurrently imaged at normal (pulse energy: 100 nJ) and low fluence levels (20, 10, and 5 nJ). The performance of the two-step denoising technique in low-fluence PAM was benchmarked against the images acquired under the normal fluence condition.

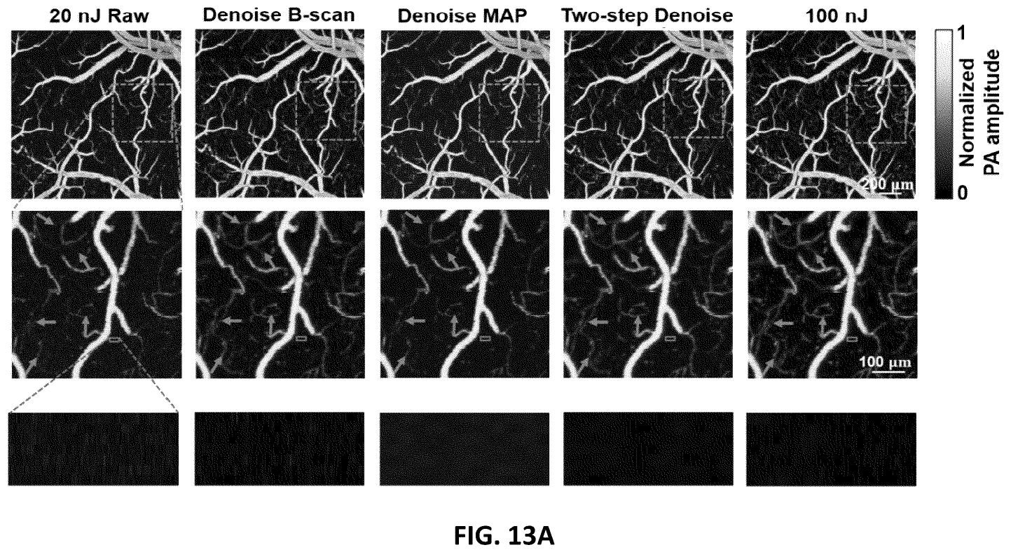

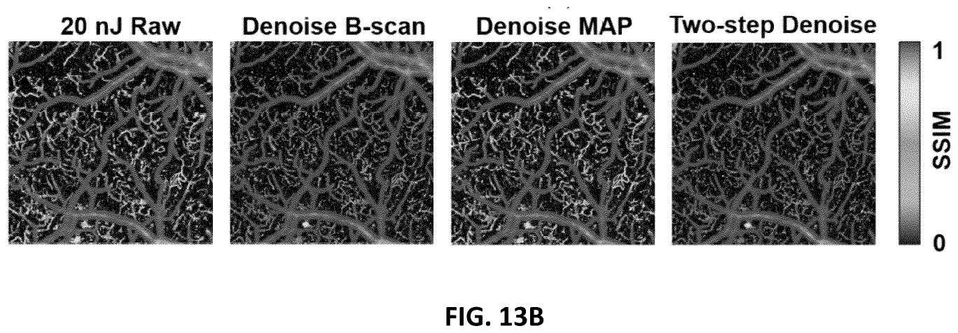

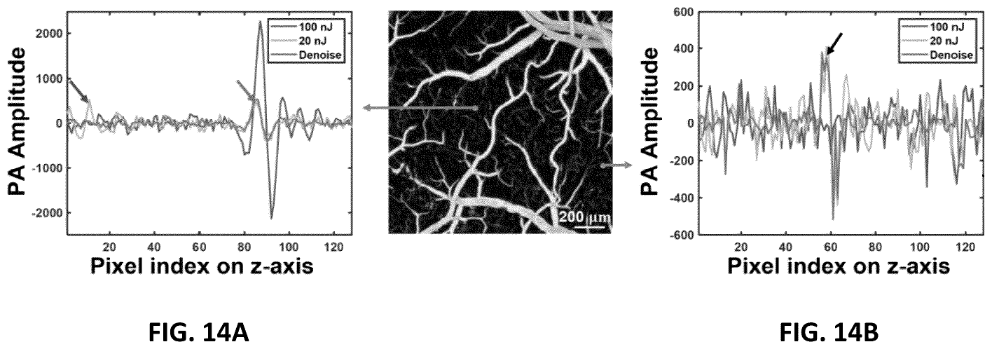

At 20% of the normal laser fluence (i.e., 20 nJ pulse energy), the two-step approach demonstrated excellent performance. As shown in FIG. 13A, the raw structural image acquired with 20 nJ laser pulses shows sparsely distributed microvessels, along with considerable non-vessel background. The sparse coding-based denoising of the B-scan (Step 1) significantly reduces the random noise in individual A-lines. Such noise removal in A-lines containing weak microvascular signals (as shown in FIG. 14A, where the noise indicated by the red arrow is comparable to the microvascular signal indicated by the green arrow) results in improved visualization of the microvessels that are barely visible in the raw image (blue arrows in FIG. 13A). However, the background in the B-scan denoised image still contains dotted patterns, likely due to ineffective suppression of the signal-like noise and/or EMI in A-lines (indicated by a black arrow in FIG. 14B). Directly applying sparse coding to denoise the raw MAP image (i.e., Step 2 only) significantly reduces the fluctuation of the background noise but does not lead to significant improvement of microvascular visualization. Combining the two steps results in more complete noise removal and a high-quality image of the microvascular structure-approaching that acquired with the normal fluence (i.e., 100 nJ).

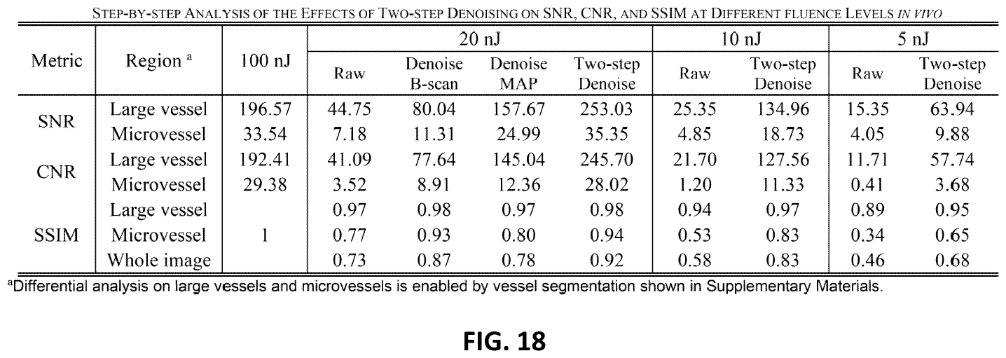

Quantitative comparison of the SNR, CNR, and SSIM values of the raw and denoised images acquired with 20 nJ laser pulses against the parameters of the image acquired with 100 nJ pulses in FIG. 18 shows three key observations. (1) The two-step denoising improves the SNR of the large vessels and microvessels by 4.9 and 5.7 times, respectively, and the CNR by 6.0 and 8.0 times, respectively. In addition, the two-step denoising improves the SSIM between the 20-nJ image and the reference image acquired at 100 nJ to 0.92, which indicates a high similarity (also shown in FIG. 13B). (2) The improvement in microvascular visualization is predominantly attributed to the B-scan denoising but not the MAP image denoising. Denoising the MAP image does not result in an appreciable increase in the microvascular SSIM (from 0.77 to 0.80). By contrast, denoising the B-scan leads to a significant increase in the microvascular SSIM (from 0.77 to 0.93). (3) Denoising the MAP image plays a dominant role in enhancing the SNR and CNR by suppressing the fluctuation of background noise.

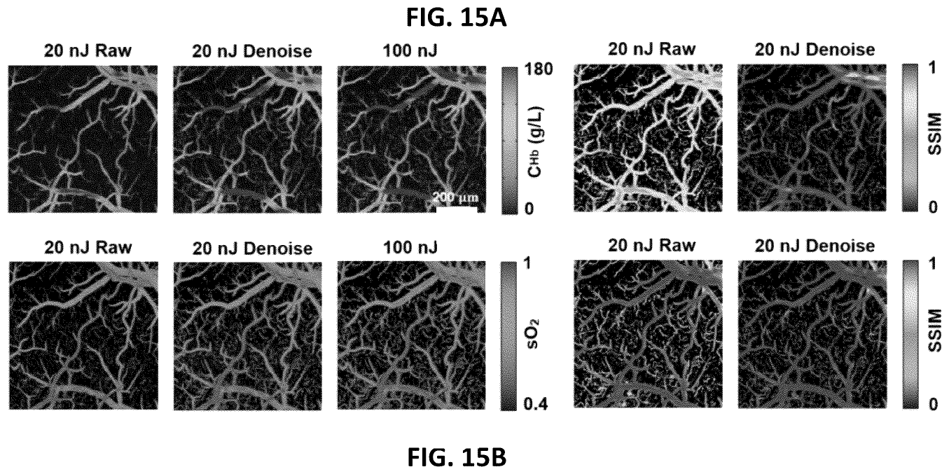

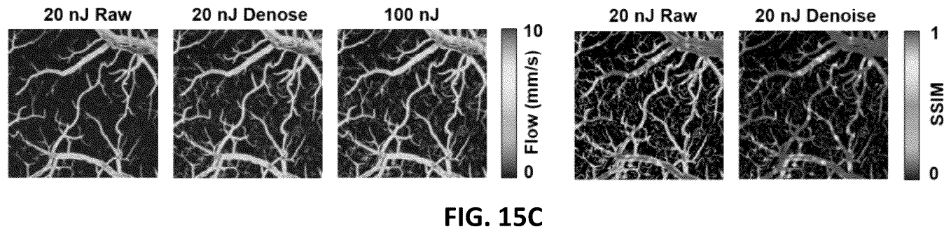

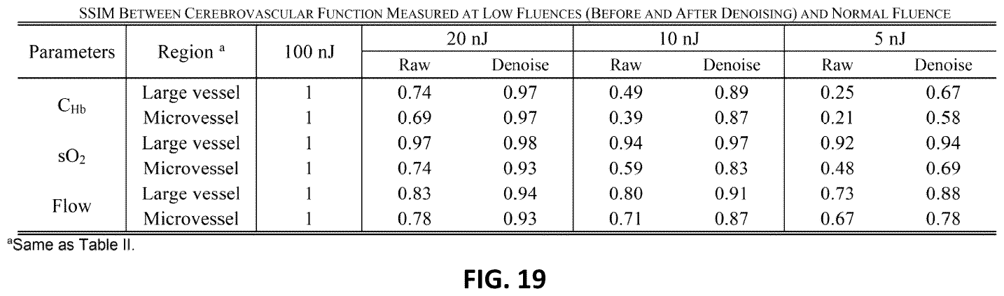

Besides the enhancement of microvascular visualization, the two-step approach also improves the accuracy of hemodynamic quantification at low-fluence levels. After denoising, the multi-parametric images acquired with 20 nJ pulses show C_{Hb}, sO₂, and flow speed values similar to those in the reference images acquired using 100 nJ pulses, as respectively shown in FIG. 15A-C. The denoising-induced improvement in quantitative accuracy is benchmarked by the SSIM between the low-fluence images (before and after denoising) and reference images, as shown in FIG. 19. For the C_{Hb} measurement, the denoising technique improves the SSIM from 0.74 to 0.97 in large vessels and from 0.69 to in microvessels. For the sO₂ measurement, the denoising not only maintains the high accuracy in large vessels (SSIM: 0.97 before denoising vs. 0.98 after denoising) but also substantially improves the accuracy in microvessels (SSIM: 0.74 before vs. 0.93 after). For the flow speed measurement, the denoising significantly improves the accuracy in both large vessels (SSIM: 0.83 before vs. 0.94 after) and microvessels (SSIM: 0.78 before vs. 0.93 after).

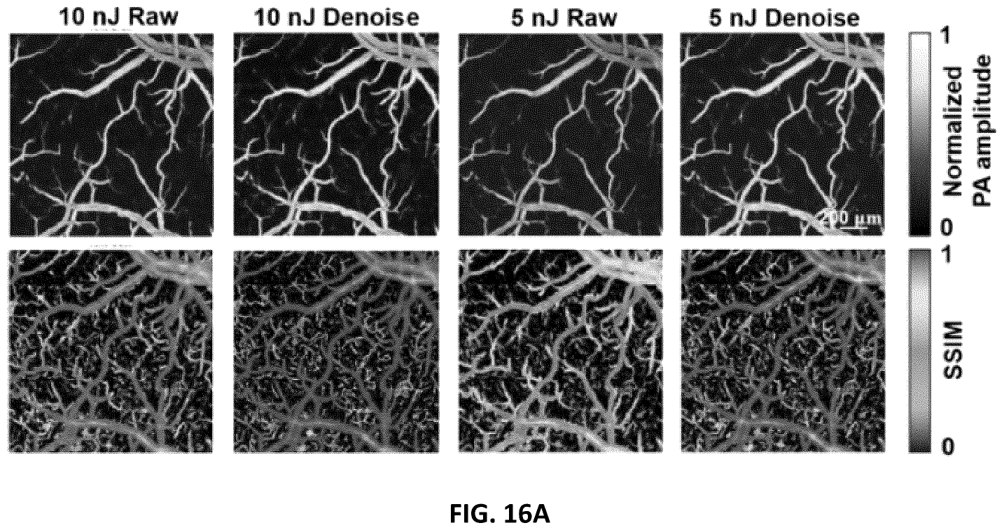

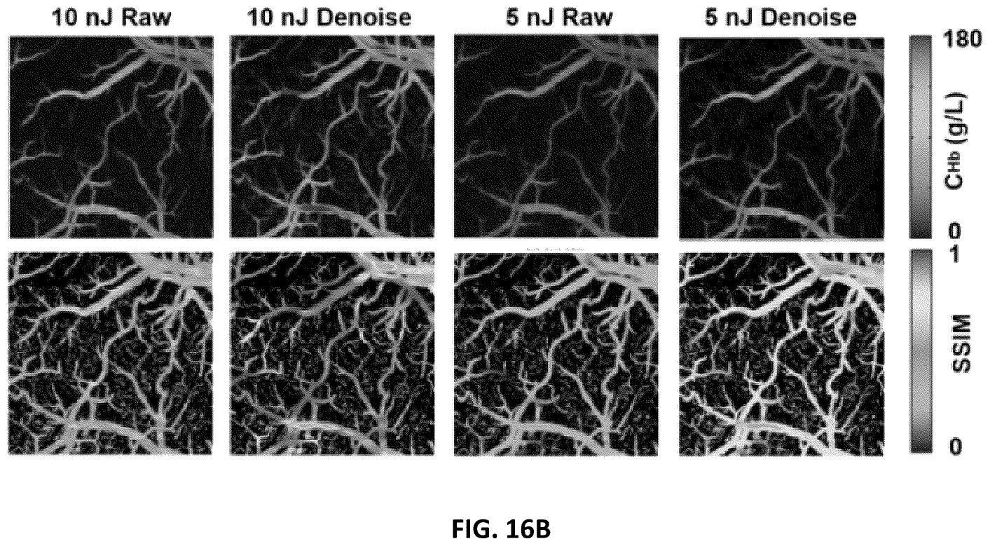

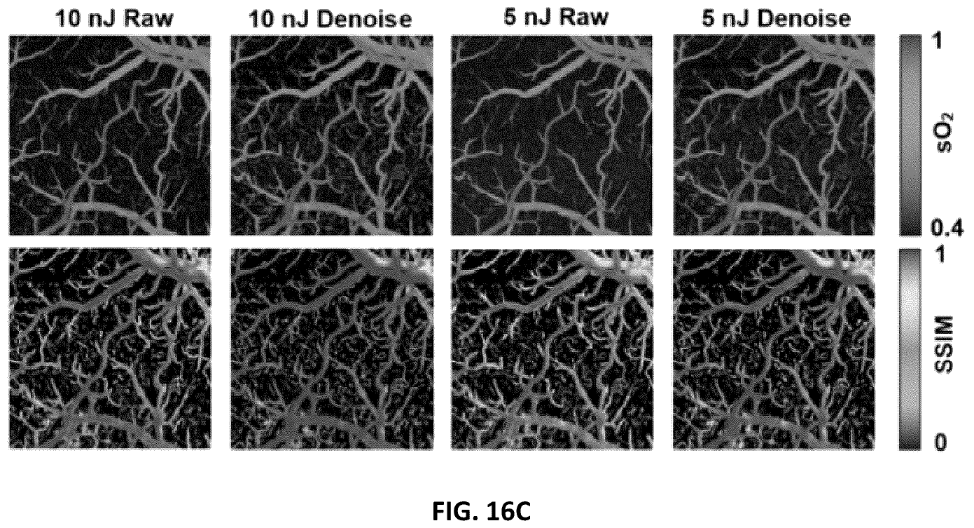

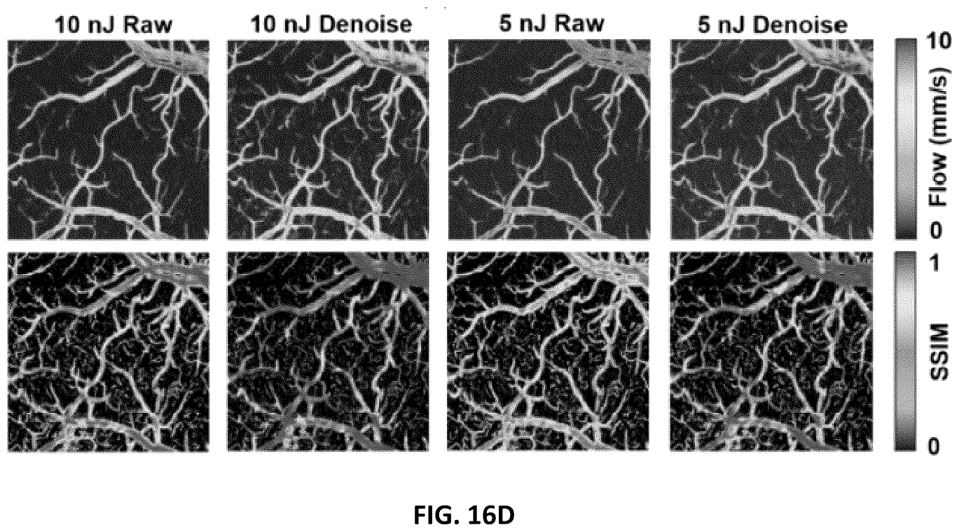

To test if this denoising technique permits a more aggressive relaxation of the fluence, the pulse energy was further reduced to 10% and 5% of the normal fluence level (i.e., 10 and 5 nJ). As shown in FIG. 16A and quantified in FIG. 18, the two-step denoising respectively improves the SNR values of large vessels and microvessels by 5.3 and 3.9 times at 10% of the normal fluence, and by 4.2 and 2.4 times at 5% of the normal fluence. Similarly, after denoising, a significant enhancement in the CNR is observed in the images acquired with 10% (5.9 and 9.4 times in large vessels and microvessels, respectively) and 5% (4.9 and 9.0 times in large vessels and microvessels, respectively) of the normal fluence. Although the image quality is significantly improved, the SSIM of microvascular structure between the denoised low-fluence images and reference image acquired with 100 nJ pulses remains considerably low (0.83 and 0.65 at 10% and 5% of the normal fluence, respectively, as shown in FIG. 18), which indicates only a partial retrieval of microvascular visualization. Similarly, this denoising technique improves the accuracy of the multi-parametric quantification. However, some SSIM values of the C_{Hb}, sO₂, and flow speed measurements remain lower than 0.9 after denoising (as shown in FIG. 19), implying that considerable errors still exist (also seen in FIG. 16B-D, respectively).

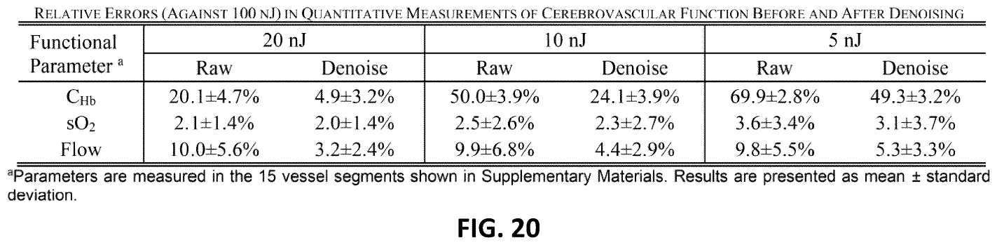

With the aid of vessel segmentation, the measurement errors are quantified for the raw and denoised C_{Hb}, sO₂, and blood flow images acquired at different fluence levels against the reference images acquired with normal fluence. As shown in FIG. 20, before denoising, the low-fluence images present considerable errors in the C_{Hb} and blood flow measurements and relatively small errors in the sO₂ measurement. At 20% of the normal fluence, the errors in C_{Hb}, sO₂, and blood flow measurements are 20.1%, 2.1%, and 10.0% before denoising and are reduced to 4.9%, 2.0%, and 3.2% after denoising, respectively. At even lower laser fluence levels, denoising can still improve quantitative accuracy, but the measurement errors remain considerably high after denoising. At 10% of the normal fluence, the denoising reduces the relative errors in C_{Hb}, sO₂, and flow measurements from 50.0%, 2.5%, and 9.9% to 24.1%, 2.3%, and 4.4%, respectively. At 5% of the normal fluence, the denoising reduces the relative errors in C_{Hb}, sO₂, and flow measurements from 69.9%, 3.6%, and 9.8% to 49.3%, 3.1%, and 5.3%, respectively.

### Conclusion and Discussion

In conclusion, a sparse coding-based two-step technique has been developed to improve the image quality and quantitative accuracy of low-fluence multi-parametric PAM. In an intravital brain imaging setting, it is shown that sequential sparse coding of the B-scans and the MAP image significantly removes the noise that accompanies the vascular signals in individual A-lines and that presents as the background fluctuation in the MAP image.

Functional quantification of C_{Hb}, sO₂, and blood flow speed is achieved by statistical, spectroscopic, and correlation analysis of PAM data, respectively. As shown by our previous study, if the photoacoustic signal is contaminated by noise, its amplitude and standard deviation, as well as the correlation of sequentially acquired A-lines, will all be affected, resulting in inaccurate quantification of these functional parameters. Effectively removing the noise while maximally preserving the amplitude and profile of the signal in low-fluence PAM images, this denoising technique offers not only improved visualization of the microvascular structure but also enhanced measurement accuracy of the microvascular function, including C_{Hb}, sO₂, and blood flow.

As an unsupervised learning strategy, the sparse coding-based denoising technique does not require a ground truth. Compared to supervised learning-based approaches, this technique is applicable in situations where the ground truth is not available.

Although demonstrated in the setting of low-fluence multi-parametric PAM, the sparse coding-based two-step denoising technique is not specific to noise type or source. It is applicable to other photoacoustic imaging systems, including high-speed PAM and deep-penetration photoacoustic tomography, where improved image quality is highly desired but often difficult to achieve due to the tight photon budget.

Using the MATLAB Parallel Computing Toolbox to process B-scans in parallel can reduce the runtime by 45% (with four parallel workers). Future implementation using a dedicated GPU can further reduce the processing time.

### Example 6—Multiparametric Photoacoustic Microscopy (MP-PAM) to Measure Oxygen-Metabolic Responses in a Live Mouse Brain Alzheimer's Disease Model

### Methods

12-16 week-old male CD-1 mice were used in this study. All animal procedures were approved by the Institutional Animal Care and Use Committee at Washington University in St. Louis. Following hair removal, a surgical incision was made in the scalp. The exposed skull was cleaned. Then, the mouse skull region over a 3×3 mm² ROI was carefully removed to expose the cortex for topical application of ATN-224. After craniotomy, the anesthetized mouse was transferred to a stereotaxic instrument. The animal body temperature was maintained at 37° C. via a heating pad and the local brain temperature was also maintained at 37° C. via a temperature-controlled water tank. Ultrasound gel was applied between the open-skull window and water tank for acoustic coupling. Following the baseline imaging, the ultrasound gel was gently removed and a solution of ATN-224 (4 μM) was applied topically. The exposed mouse cortex was treated with the SOD1 inhibitor solution for 80 min. Then, the ROI was covered again with ultrasound gel and subjected to post-treatment MP-PAM imaging. For 2P-FLIM imaging of the live mouse brain, the same pre-imaging procedure was used, but in this case for 12-week-old male C57BL/6 mice. After surgery, the mice were transferred to a stereotaxic frame at the side of the Zeiss LSM-780 microscope. An objective converter was used to convert the inverted LSM-780 to an upright configuration. A 20×/NA 0.85 water immersion objective lens was coupled to the converter and gently moved to the cranial window for FLIM imaging.

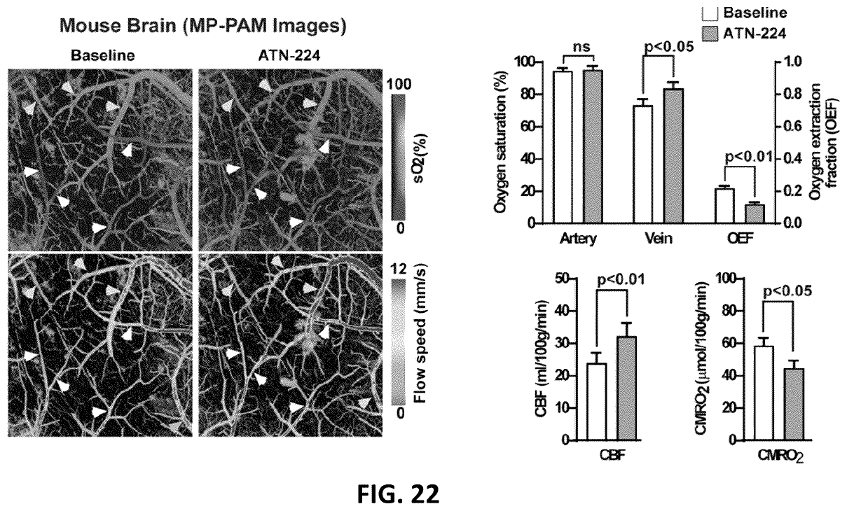

For MP-PAM assays, vessel segmentations and quantitative analysis were done following the protocols described in the present disclosure. The paired t-test was used for comparing the cerebral hemodynamics and oxygen metabolism before and after the application of the compounds, which is shown in FIG. 22. A p-value of less than 0.05 was considered statistically significant.

### Results

Multiparametric photoacoustic microscopy (MP-PAM) was applied to measure oxygen-metabolic responses in the live mouse brain. MP-PAM imaging through a cranial window enables simultaneous, high-resolution imaging of total hemoglobin concentration (C_{Hb}), oxygen saturation of hemoglobin (sO₂), and cerebral blood flow. This approach revealed that ATN-224 caused significant increases in venous sO₂, without changes in cerebral blood flow (CBF; FIG. 22). These hemodynamic responses to ATN-224 were accompanied by decreases in both oxygen extraction fraction (OEF) and the cerebral metabolic rate of oxygen (CMRO₂; FIG. 22).

### Example 7—High-Speed Multi-Parametric Photoacoustic Microscopy of Cerebral Hemodynamic and Metabolic Responses to Acute Hemodilution

### Summary

The ability of hemodilution to improve vascular circulatory impairment has been demonstrated. However, the effects of acute hemodilution on cerebral hemodynamics and oxygen metabolism have not been assessed at the microscopic level, due to technical limitations. To fill this void, developed a photoacoustic microscopy system has been developed, which enables high-speed imaging of blood hemoglobin concentration, oxygenation, flow, and oxygen metabolism in vivo. The system performance was examined in both phantoms and the awake mouse brain. This new technique enabled wide-field (4.5×3 mm²) multi-parametric imaging of the mouse cortex at 1 frame/min. Narrowing the field of view to 1.5×1.5 mm² allowed dynamic imaging of the cerebral hemodynamic and metabolic responses to acute hypervolemic hemodilution at 6 frames/min. Quantitative analysis of the hemodilution-induced cerebrovascular responses over time showed rapid increases in the vessel diameter (within 50-210 s) and blood flow (50-210 s), as well as decreases in the hemoglobin concentration (10-480 s) and metabolic rate of oxygen (20-480 s) after the acute hemodilution, followed by a gradual recovery to the baseline levels in 1440 s. Providing comprehensive insights into dynamic changes of the cerebrovascular structure and function in vivo, this technique opens new opportunities for mechanistic studies of acute brain diseases or responses to various stimuli.

### DETAILED DESCRIPTION

As a potential therapy for brain ischemia, hemodilution has been suggested to increase cerebral blood flow (CBF) and improve circulatory impairment, but the neuroprotective effect remains unclear. Thus, studying how hemodilution influences cerebral hemodynamics and metabolism has considerable translational value. Widely used in experimental and clinical settings, acute normovolemic hemodilution reduces the demand for homologous blood. An alternative that is quicker and easier to implement is acute hypervolemic hemodilution, where saline or hydroxyethyl starch is infused without blood removal. However, the acute effect of hypervolemic hemodilution necessitates high-speed imaging to capture the dynamic responses.

Enabling simultaneous imaging of hemoglobin concentration (C_{Hb}), blood oxygenation (sO₂), and CBF at the microscopic level, multi-parametric photoacoustic microscopy (PAM) is ideally suited to study the effect of hemodilution on brain hemodynamics. Further, combining these functional measurements enables the study of hemodilution-induced changes in the oxygen extraction fraction (OEF) and cerebral metabolic rate of oxygen (CMRO₂). However, existing multi-parametric PAM techniques do not have sufficient speeds for studying the rapid hemodynamic and oxygen-metabolic changes caused by acute hypervolemic hemodilution. Recently, several methods have been developed to improve the speed of PAM. By using a water-immersible micro-electro-mechanical system (MEMS), functional imaging of the mouse brain with an A-line rate of 100 kHz has been realized. With the use of a water-immersible polygon-scanning mirror, the A-line rate of PAM has been improved to 1 MHz. Although encouraging, these techniques are not readily applicable for correlation-based CBF quantification due to their high B-scan rates. To improve the speed while maintaining the relatively low B-scan rate of ˜1 mm/s needed for CBF measurements, an optical-mechanical hybrid-scan approach has been developed to simultaneously acquire multiple B-scans. However, the small ultrasonic detection area (40 μm in diameter) significantly limits the range of optical scanning and the improvement in imaging speed. Exploiting the line-shaped focus (76 μm×4.5 mm) of a cylindrically focused ultrasonic transducer, multi-parametric PAM over a large field of view (1 mm×4.5 mm) can be completed within a second. It is worth noting that the high-speed system reported by Zhong et al. operates in the transmission mode and thus is not suited for brain imaging in vivo. Moreover, existing high-speed PAM systems are mostly based on two-dimensional (2D) raster scanning within a horizontal plane, which cannot address the out-of-focus issue caused by the uneven surface of the mouse brain, resulting in a poor image resolution and low signal-to-noise ratio (SNR).

In this example, a high-speed multi-parametric PAM system with real-time contour scanning capacity was developed to study the dynamic responses of the cerebrovascular structure, function, and oxygen metabolism to acute hypervolemic hemodilution in the awake mouse brain. High-speed acquisition of multi-parametric images at two different spatial scales (i.e., 4.5×3 mm² and 1.5×1.5 mm²) was demonstrated. Combining the hardware innovation with the vessel segmentation, hypervolemic hemodilution-induced changes in cerebrovascular diameter, C_{Hb}, CBF, sO₂, OEF, and CMRO₂ were comprehensively characterized.

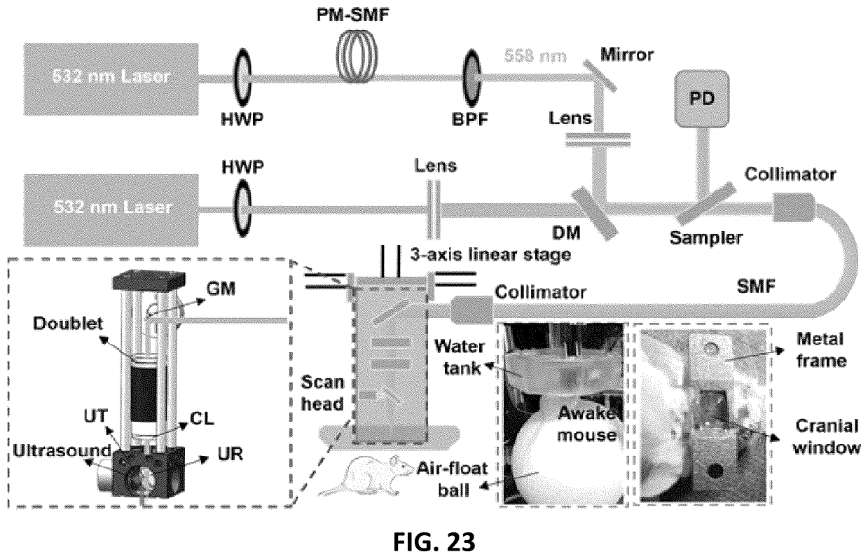

As shown in FIG. 23, the high-speed multi-parametric PAM uses two ytterbium-doped fiber lasers (GLPM-10-Y13, IPG Photonics; wavelength, 532 nm). After going through a half-wave plate (HWP; WPH05M-532, Thorlabs), one of the laser beams is coupled into a 10-m-long polarization-maintaining single-mode fiber (PM-SMF; HB450-SC, Fibercore) for the stimulated Raman scattering-based wavelength conversion. Then, a band-pass filter (BPF; FB560-Thorlabs) is used to isolate the 558-nm Stokes component. The 558-nm Stokes beam and the 532-nm output from the other laser are separately expanded by two lens pairs and then combined by a dichroic mirror (DM; T550lpxr, Chroma).

The combined beam is collimated by a fiber collimator (CFC-11X-A, Thorlabs) and coupled into the scan head of PAM through a 2-m-long regular single-mode fiber (SMF; P1-460B-FC-2, Thorlabs). Before the fiber coupling, ˜5% of the combined laser light is tapped off a beam sampler (Sampler; BSF10-A, Thorlabs) and detected by a high-speed photodiode (PD; FDS100, Thorlabs) to compensate for possible laser fluctuation. In the scanning head (left-hand dashed-boxed inset in FIG. 23, the dual-wavelength beam coming out of the SMF is collimated by a fiber collimator (CFC-11X-A, Thorlabs), reflected by a two-axis galvanometer scanner (GM; 6215HSM40B, Cambridge Technology) and focused by a doublet (AC127-025-A, Thorlabs) onto the imaging object through a correction lens (CL; KPX561, Newport) and an ultrasonic reflector (UR; LOT-18928, VWR; material, glass; thickness, 0.14 mm; size, 5×5 mm² square). The UR transmits the laser beam but reflects the photoacoustic wave for detection by a horizontally positioned weakly focused ultrasonic transducer (UT; customized by the Ultrasonic Transducer Resource Center at the University of Southern California; focal zone, 250 μm; center frequency, 30 MHz; 6-dB bandwidth, 50%). A hollow cube is machined to house the UR and UT, and the cube with UR and UT is immersed in a water tank. The scanning head is mounted on a three-axis linear stage (PLS-85, PI miCos) for raster scanning in the x-y plane while simultaneously adjusting the optical-ultrasonic dual foci in the z-direction. For awake-brain imaging, a head-restraint apparatus for PAM is adopted. As shown in the middle dashed-boxed inset of FIG. 23, the mouse is placed on a polystyrene ball-based treadmill beneath the water tank. At least two weeks prior to the imaging, a 3D-printed metal frame is firmly cemented to the surface of the mouse skull for head fixation, leaving the cranial window exposed for PAM imaging access (right-hand dashed-boxed inset in FIG. 23).

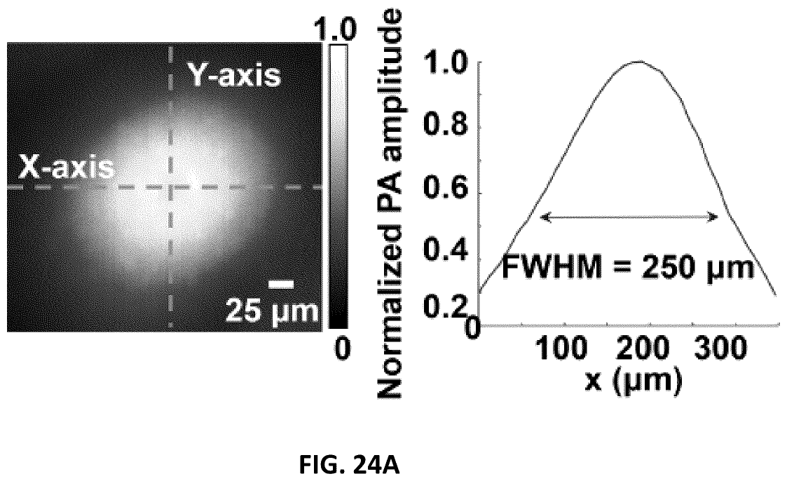

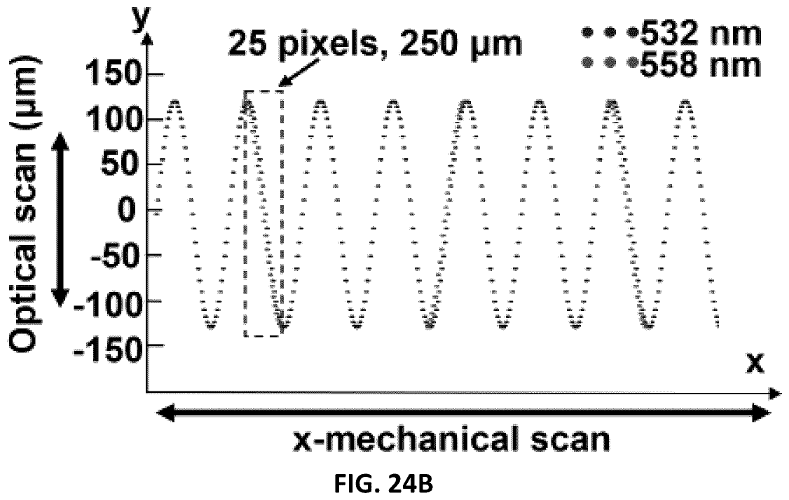

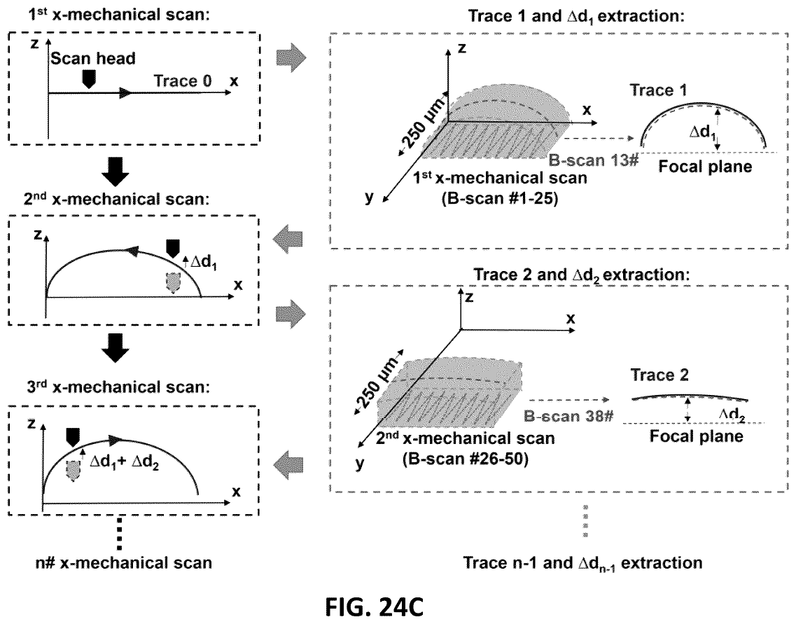

To overcome the limitation due to the small detection area of the tightly focused UT (40 μm in diameter), a weakly focused UT is applied to increase the focal diameter, enabling the simultaneous acquisition of more B-scans. The focal diameter of the UT is experimentally assessed by a 2D optical scan of a black tape (FIG. 24A). The shape of the acoustic focal zone is characterized as a circular region with relatively high photoacoustic amplitudes. Gaussian fitting yields an acoustic focal diameter of 250 μm, which is estimated as the full width at half-maximum (FWHM) value.

As shown in FIG. 24B, the GM steers the optical focus along the y-axis within the 250-μm ultrasonic focus at a round-trip rate of 12 kHz. Concurrently, the x-axis linear stage mechanically translates the scanning head at 1 mm/s for flow speed quantification, during which the 532-nm and 558-nm pulses alternately excite the imaging object to produce dual-wavelength A-line pairs for sO₂ calculation. Since dense sampling is not required for sO₂ measurements, the 558-nm pulses are generated only at every five A-lines of the optical scan. With a 600-kHz laser repetition rate, 25 B-scans can be simultaneously acquired by using the optical-mechanical hybrid scan.

To address the out-of-focus issue due to the uneven surface of the mouse brain, a real-time contour scanning technique was developed and adopted. By exploiting the similarity of brain surface contours between adjacent mechanical scans along the x-axis, the surface contour can be extracted in real-time to guide the next x-mechanical scan. As shown in FIG. 24C, first, the scan head travels along the x-axis (i.e., the horizontal line denoted as Trace 0) to acquire the first x-mechanical scan, which consists of 25 B-scans (#1-25). The data acquired in B-scan #13 is extracted for surface curve fitting, which is denoted as Trace 1. Here, Δd1 is calculated from the difference between the extracted trace and the focal plane to guide the movement of the z-axis stage for the second x-mechanical scan. Between the first x-mechanical scan and the second x-mechanical scan, the y-axis stage moves with a step size of 250 μm. When the second x-mechanical scan (i.e., B-scan #26-50) starts, the z-axis stage moves by Δd1 in the reverse direction. Similarly, Trace 2 and Δd2 are extracted by the middle B-scan #38. Due to the function of the contour scan, the curve fitting of Trace 2 is relatively flat and the next contour scan can be fine-tuned. Thus, during the third x-mechanical scan, the z-axis stage moves again according to the sum of Δd1 and Δd2, and the procedure is repeated until the scan is complete.

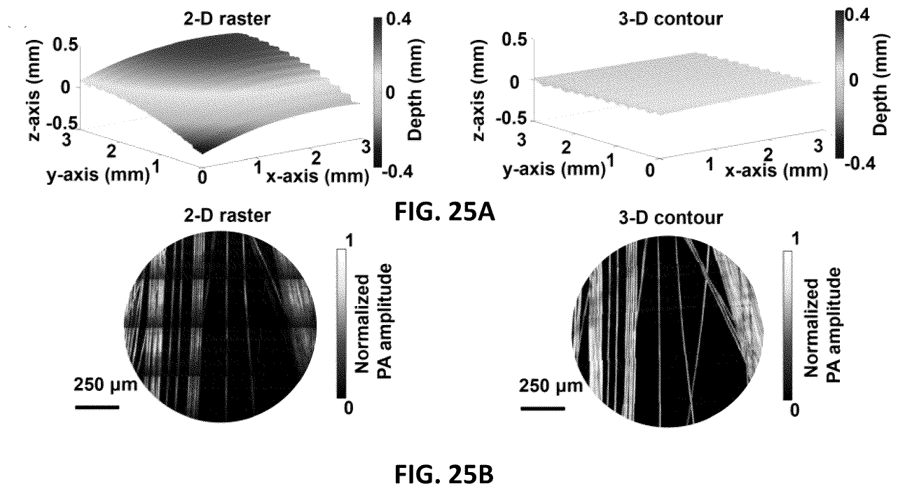

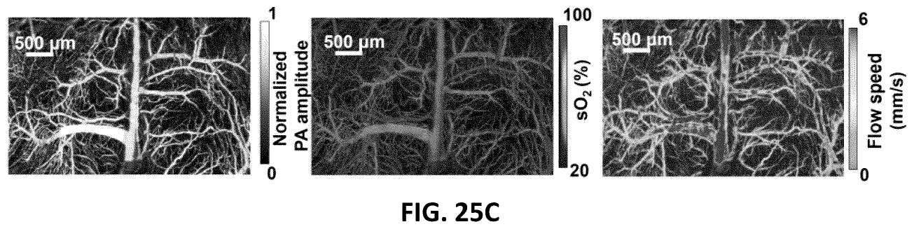

To validate the utility of the 3D optical-mechanical contour scan, a phantom made from a black tape attached to the surface of a 20-mm diameter plastic ball was imaged (FIG. 25A). It is observed that, in the case of 2D raster scanning, more and more areas are out of focus with the expansion of the scanning range. In contrast, using 3D contour scanning, the surface contour remains close to the focal plane with a very small standard deviation in depth (25 μm). Next, the black tape on the ball surface was replaced with a cluster of 7-μm carbon fibers (S-CF706-T700, CST) to examine the resolution. As shown in FIG. the image acquired with a 2D raster scan (left) shows relatively poor resolution and low SNR (3.3 dB), while that acquired with the 3D contour scan (right) has a higher SNR (6.7 dB), by addressing the out-of-focus issue.

We further tested the in vivo performance of the system in the awake mouse brain. The microvascular structure, sO₂, and blood flow over a large area (4.5×3 mm²) in the mouse cortex were simultaneously imaged. As shown in FIG. 25C, the high-speed 3D contour scanning enables high-quality cortex-wide microvascular imaging. Moreover, the sO₂ and flow images show the ability of this multi-parametric PAM system for quantitative measurements of microvascular function, and the total acquisition time was ˜1 min.

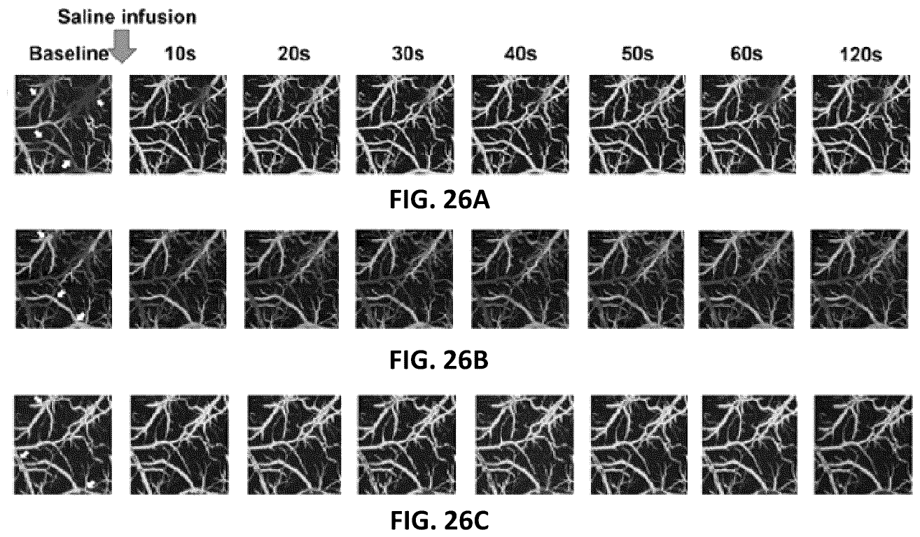

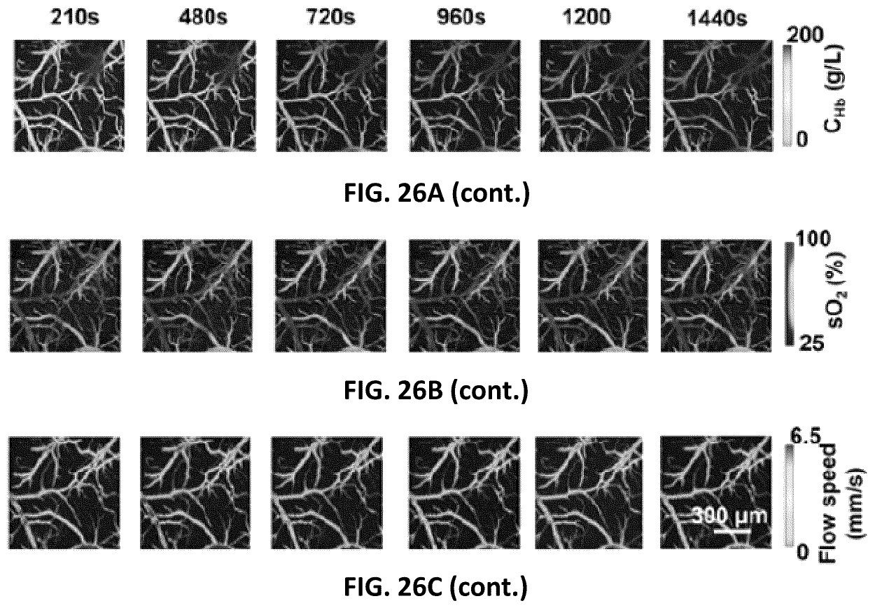

After characterizing the performance of the high-speed multi-parametric PAM, cerebral hemodynamic and oxygen-metabolic responses of the awake mouse brain to acute hypervolemic hemodilution were studied. Male CD-1 mice (9 weeks old, Charles River Laboratories) were used. The mouse model of acute hypervolemic hemodilution was based on saline infusion. Before imaging, the tail vein was cannulated to create a venous line. During imaging, hypervolemic hemodilution was achieved by a rapid saline infusion (10 ml/kg, 1 ml/min) through the venous line. Multi-parametric PAM was performed on a cortical region of interest (1.5×1.5 mm²) continuously for 24 min at 6 frames/min. Time-lapse recording showed the dynamic changes in the cerebral vasculature following the saline infusion (FIG. 26). Immediately after the infusion, the mouse brain showed a rapid decrease in the C_{Hb} (10-50 s as indicated by the arrows in FIG. 26), an increase in the venous sO₂ (20-210 s in FIG. 26B), and an elevation in the blood flow speed (10-480 s in FIG. 26C). These acute changes were followed by a gradual recovery.

This experiment was repeated in four mice, and the vessel segmentation was performed for quantitative analysis. The completion of the saline infusion was set as the time zero and analyzed the acute hemodilution-induced percentage changes over time for the vessel diameter, C_{Hb}, CBF, sO₂, OEF, and CMRO₂.

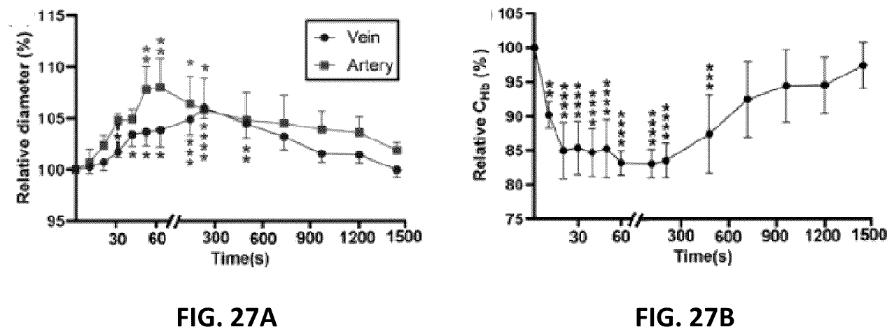

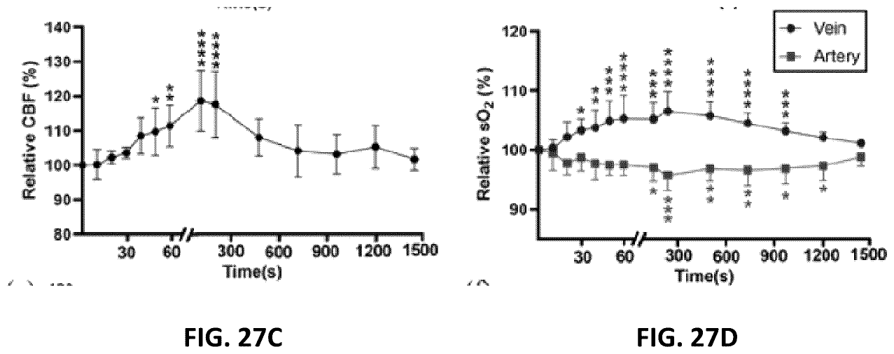

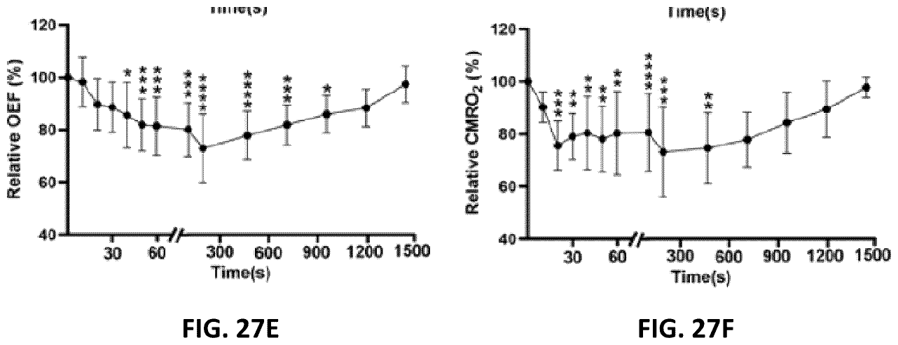

Structurally, as shown in FIG. 27A, the diameters of both arteries and veins showed continuous and significant vasodilation starting at 40 s after the saline infusion, reaching maximum relative changes of 108.0±5.6% at 60 s for the arteries and 106.0±2.1% at 210 s for the veins. Then, the diameters of the vessels began to reduce, gradually returning to the baseline levels (101.9±1.6% and 99.9±1.7% for arteries and veins, respectively) within 1440 s. According to the two-way repeated-measures analysis of variance (ANOVA), time was a significant factor in determining the vessel diameter [F (13, 78) 7.767, p<0.001]. However, the vessel types (artery versus vein) were not a significant factor to influence their diameters [F (1, 6) 1.036, p=0.348]. There was no interaction between the vessel type and time [F (13, 78) 0.845, p=0.612].

Functionally, the C_{Hb} dropped significantly right after the saline infusion, from 90.2±1.9% at 10 s to 87.4±5.7% at 480 s [FIG. 27B]. Moreover, the blood flow speed increased in response to the acute hemodilution, which together with the vasodilation resulted in a significant increase in the CBF that occurred after the hemodilution and peaked at 118.9±8.8% of the baseline at 120 s [FIG. 27C]. The observations in the mouse brain echoed a human study using positron emission tomography (PET), which showed a reduced C_{Hb} and an increased CBF in response to hemodilution. After 210 s, the CBF slowly recovered to a near-baseline level of 101.7±3.2% at 1440 s. The venous sO₂ showed a statistically significant increase to a maximum of 106.5±3.3% from 30 s to 210 s, which is likely due to the increased CBF and O₂ supply, without an accompanying increase in brain O₂ uptake. Meanwhile, the arterial sO₂ decreased moderately over time, to 95.7±2.6% at 210 s, as shown in FIG. 27D. The significant increase in the venous sO₂, along with the very moderate change in the arterial sO₂, led to an OEF decease to 73.0±13.1% at 210 s, as shown in FIG. 27E. Between 480 s to 1440 s, the OEF gradually recovered to a near-baseline level.

Metabolically, a statistically significant decrease in the regional CMRO₂ was observed right after the acute hemodilution, reaching its minimum of 73.2±17.1% at 210 s [FIG. 27F]. The data indicated that the total CMRO₂ in the region of interest decreased despite a significant increase in the CBF. Similar to the structural and functional parameters, CMRO₂ also showed a gradual recovery, returning to the normal metabolic rate at around 1440 s.

In summary, a high-speed multi-parametric PAM technique with 3D contour scanning has been developed for dynamic imaging of the acute hemodilution-induced changes in cerebrovascular structure, function, and oxygen metabolism in the awake mouse brain. By using a weakly focused ultrasonic transducer, 25 B-scans can be simultaneously acquired with a single x-mechanical scan. Moreover, real-time adjustment of the optical-acoustic foci enables high-resolution imaging over the uneven mouse cortex without the out-of-focus issue. The system performance was examined in both phantoms and in vivo. Taken at 6 frames/min over a cortical region of 1.5×1.5 mm², multi-parametric PAM images revealed dynamic changes in cerebral hemodynamics and oxygen metabolism in response to acute hemodilution. Statistical analysis of the results showed transient but significant responses, including vasodilation, reduced C_{Hb}, increased CBF, and decreased OEF and CMRO₂, right after the acute challenge. The high speed, large range, and multi-parametric insights offered by this new PAM technique open new opportunities for mechanistic studies of the dynamic aspects of brain diseases and responses to stimuli.

### Example 8—Thin-Film Optical-Acoustic Combiner Enables High-Speed Wide-Field Multi-Parametric Photoacoustic Microscopy in Reflection Mode

### Summary

Multi-parametric photoacoustic microscopy (PAM) is uniquely capable of simultaneous high-resolution mapping of blood oxygenation and flow in vivo. However, its speed has been limited by the dense sampling required for blood flow quantification. To overcome this limitation, a high-speed multi-parametric PAM system has been developed, which enables the simultaneous acquisition of ˜500 densely sampled B-scans by superposing the rapid optical scanning across the line-shaped focus of a cylindrically focused ultrasonic transducer over the conventional mechanical scan of the optical-acoustic dual foci. An optical-acoustic combiner (OAC) is designed and implemented to accommodate the short working distance of the transducer, enabling convenient confocal alignment of the dual foci in reflection mode. A resonant galvanometer (GM) provides stabilized high-speed large-angle scanning. This new system can continuously monitor microvascular blood oxygenation (sO₂) and flow over a 4.5×3 mm² area in the awake mouse brain with high spatial and temporal resolutions (6.9 μm and 0.3 Hz, respectively).

### DETAILED DESCRIPTION

Capable of high-resolution structural, functional, molecular, and metabolic imaging in vivo, photoacoustic microscopy (PAM) has shown considerable promise in basic and translational research. Further, the recent development of multi-parametric PAM enables simultaneous imaging of microvascular blood oxygenation (sO₂) and flow speed. However, the dense spatial sampling (i.e., small scanning step size) required for blood flow quantification significantly limits the speed of multi-parametric PAM and prevents dynamic assessments of microvascular function and tissue oxygen metabolism.

Currently, in multi-parametric PAM, the blood flow is quantified by analyzing the flow-induced decorrelation between adjacent A-line signals. Although showing robust performance in measuring flow speeds across a wide range that is physiologically relevant (i.e., 0.18-21 mm/s) and in blood vessels of different diameters, this method requires dense sampling (i.e., ˜0.5 μm step size) in order to accurately extract the correlation decay constant. As a result, the B-scan rate is limited to −1 mm/s, which prevents the use of existing methods developed for high-speed data acquisition in conventional PAM and presents a barrier to improving the speed of multi-parametric PAM. Although other methods have been developed or adopted for blood flow measurements in PAM, they have important limitations. Dynamically tracking the movement of individual blood cells is widely used for measuring microvascular flow, but this method is not readily applicable to flow quantification in large vessels where blood cells typically do not travel in single file. Dual-pulse photoacoustic flowmetry based on the Grüneisen relaxation effect shows the promise of single-shot-based flow measurements; however, an excessive average (i.e., 100 times) is required, which significantly limits the imaging speed.

To improve the speed of multi-parametric PAM, an optical-mechanical hybrid scan strategy has been utilized to simultaneously acquire multiple B-scans while maintaining the dense sampling required for the correlation-based flow measurement. The original demonstration used a spherically focused ultrasonic transducer. Although the tight acoustic focus offered high sensitivity, the small acoustic focal zone limited the optical scanning range and thus the number of B-scans that could be simultaneously acquired. To overcome this limitation, the spherically focused transducer was replaced with a cylindrically focused transducer. However, the short working distance (6 mm) of the cylindrically focused transducer, which is required to achieve tight focus along one dimension for sufficient sensitivity, challenges the integration of optical excitation and ultrasonic detection in reflection mode. Although multiple different strategies have been developed to integrate light and ultrasound in PAM, none can be readily applied to meet our development goal. For example, an acoustic reflector with a central opening for optical excitation was previously developed as an optical-acoustic combiner (OAC) for PAM; however, that design is not compatible with the short working distance of the cylindrically focused transducer. The same difficulty is shared by other types of OACs that transmit light and reflect ultrasound, including an optically transparent acoustic reflector (e.g., a glass plate), a dual-prism cube with a thin layer of silicone oil in between, and a single prism with an optical index-matching fluid.

Here, a new small-footprint OAC is reported, which has enabled the implementation of high-speed multi-parametric PAM in reflection mode. Combining a high-repetition-rate pulsed laser, a high-speed resonant galvanometer (GM), a cylindrically focused transducer, and the new OAC, the new multi-parametric PAM has achieved a 112-fold improvement in imaging speed over our previous system—enabling simultaneous imaging of C_{Hb}, sO₂, and blood flow over an extended area of 4.5×3 mm² in 3 s. The acquisition time can be further reduced to 0.5 s if the flow measurement (and thus dense sampling) is not required. The utility of the high-speed wide-field multi-parametric PAM has been demonstrated in the awake mouse brain by monitoring the dynamic changes of cerebrovascular blood oxygenation and flow in response to hypoxic challenges.

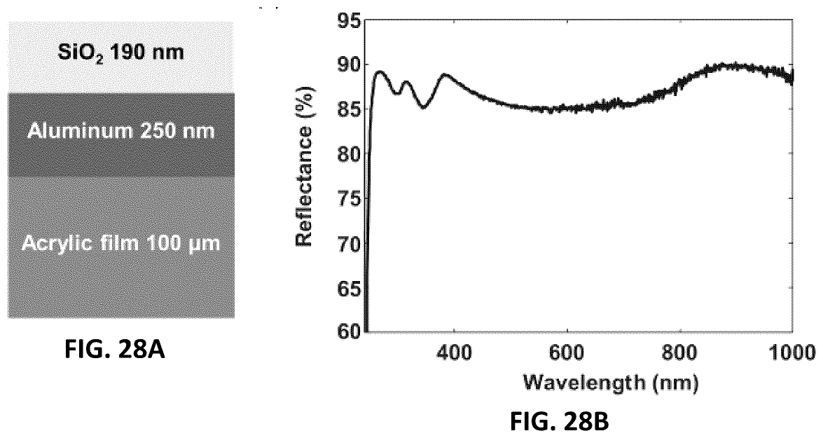

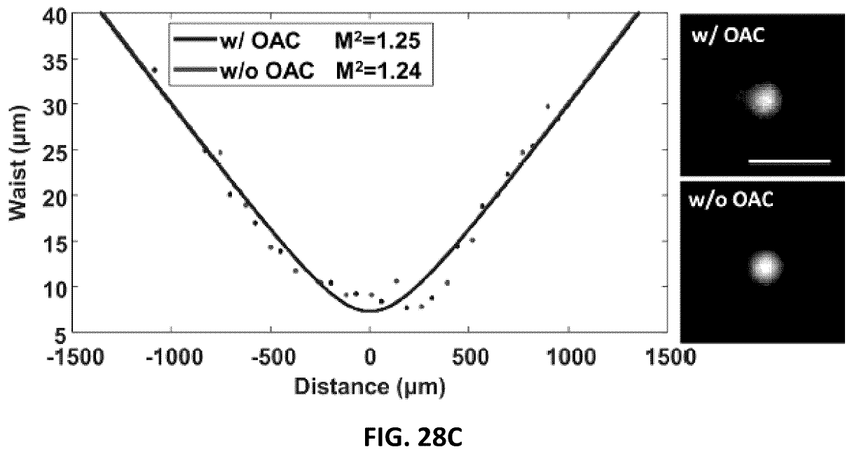

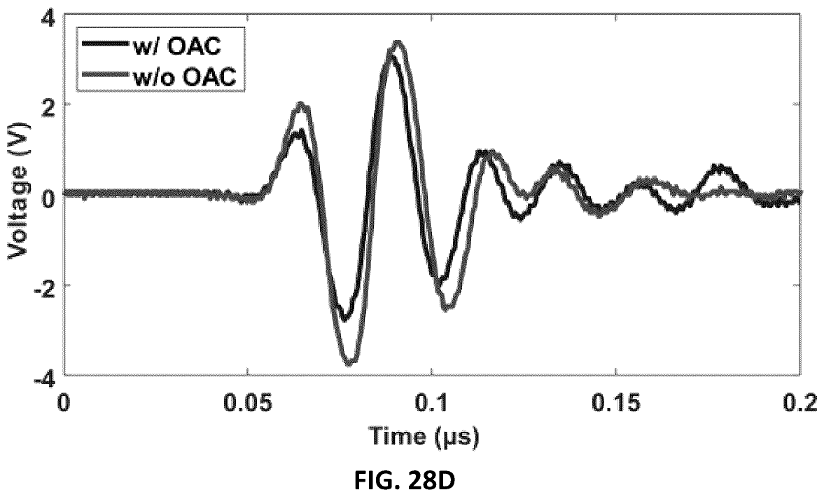

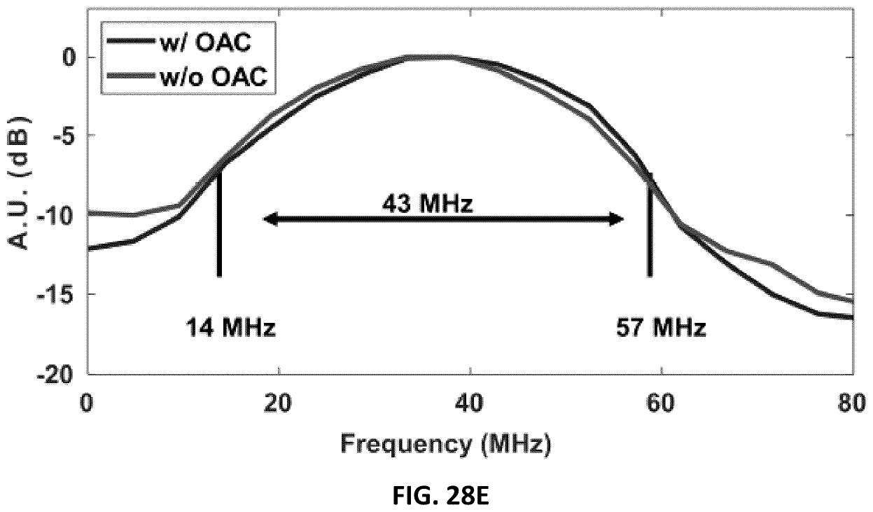

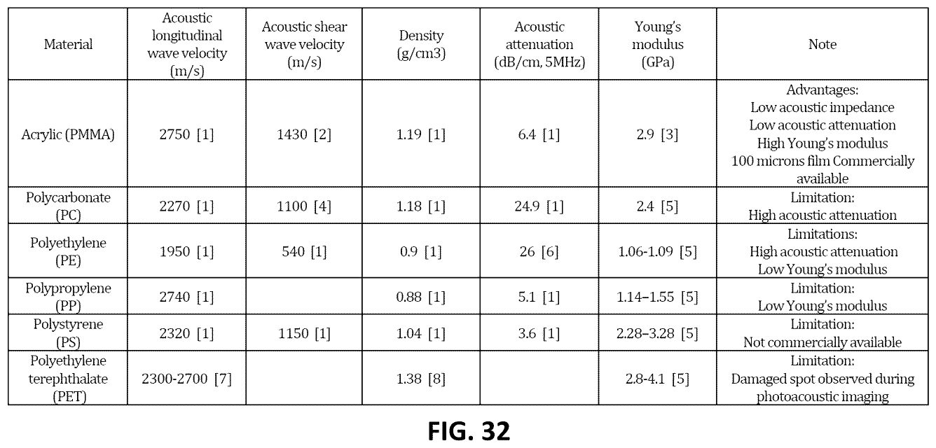

As shown in FIG. 28A, the OAC is fabricated on a 100-μm-thick acrylic film. Then, a 250-nm-thick layer of aluminum is coated on the film to achieve light reflection in the visible spectral range. Finally, a 190-nm-thick layer of SiO₂ is coated on the top to protect the delicate aluminum coating during experiments in a water environment. In the PAM system, a 3D-printed mount holds the OAC between the cylindrically focused transducer and the imaging target at a 45° angle to the illumination beam. The focused laser excitation beam is reflected by the aluminum layer onto the imaging target, while the generated ultrasonic wave back-propagates through the OAC and is detected by the transducer. PMMA acrylic film is selected for its high optical transparency in the visible spectral range, low acoustic attenuation, stability, and commercial availability (see FIG. 32). With a relatively low density (1.19 g/cm³) and acoustic velocity (2.8×10³ m/s), the acrylic film has an acoustic impedance similar to the coupling medium (i.e., water) which makes it an ideal substrate for the OAC to minimize acoustic reflection. Since the combined thickness of the two coating layers is much smaller than the ultrasonic wavelength, they are transparent to light-generated ultrasonic waves.

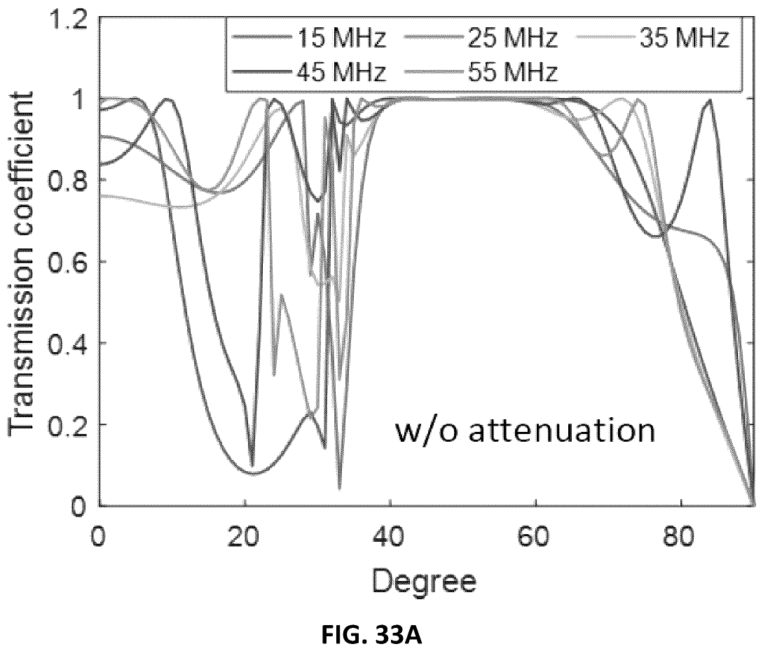

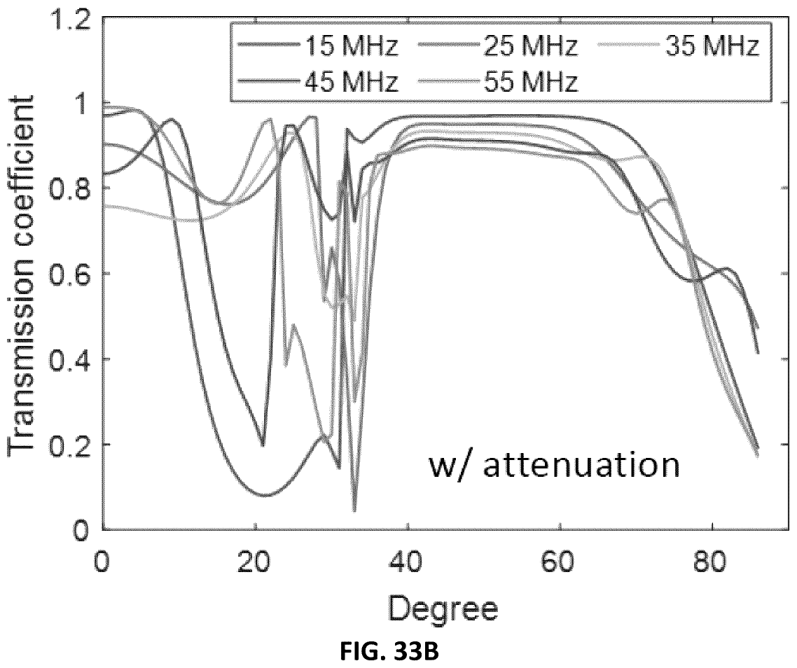

The performance of the new OAC was evaluated by comprehensively quantifying its influence on PAM's optical excitation and ultrasonic detection. The reflection spectrum of the OAC was measured using a commercial spectrophotometer (Varian Cary 50 Bio), showing a high reflectance at visible wavelengths [FIG. 28B]. To examine its influence on light focusing, we measured the beam quality factor (M2) with or without the OAC. As shown in FIG. 28C, the focus, the beam waist, and M2 remain largely the same under both conditions, indicating that no apparent optical aberration is induced. Moreover, we performed a pulse-echo experiment to quantify the influence of the OAC on the propagation of ultrasonic waves. A pulser-receiver (5073PR, Olympus) drove the cylindrically focused transducer, and the generated ultrasonic wave was reflected by a glass slide and then received by the same transducer. The same measurement was repeated with the OAC inserted between the pulser-receiver and the glass slide. As shown in FIG. 28D, the peak-to-peak amplitudes of the echo signals with and without the OAC are 5.9 V and 7.1 V, respectively, which indicates that the one-way acoustic attenuation of the OAC is <9%. This attenuation is almost completely due to shear-wave attenuation in the film rather than the reflection at the surfaces. The experimental result is consistent with the simulation result in FIG. 33. Further, Fourier analysis of the pulse-echo data shows that the normalized frequency spectra of the echo signals in the presence or absence of the OAC are nearly identical, showing a 6-dB bandwidth of 43 MHz with the lower boundary at 14 MHz and the higher boundary at 57 MHz [FIG. 28E]. Together, these results suggest that the OAC has minimal influence on optical excitation and ultrasonic detection.

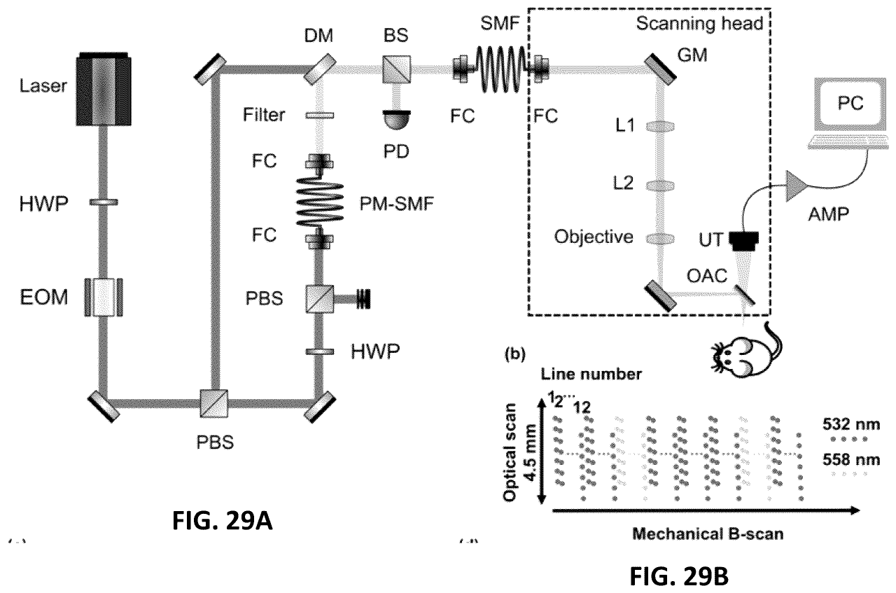

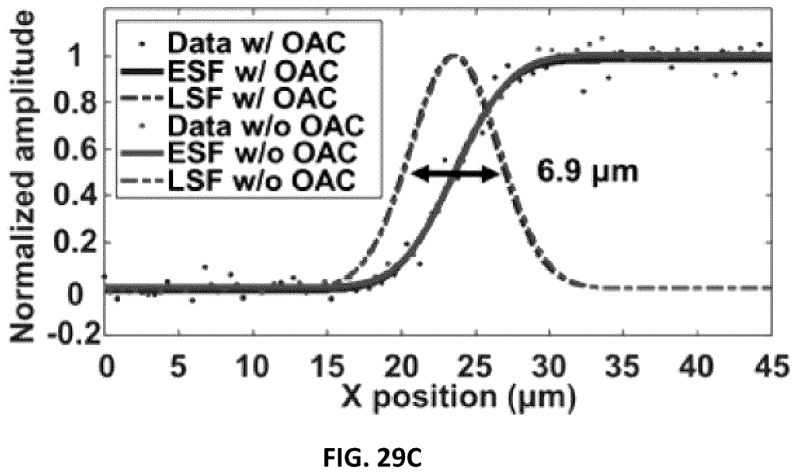

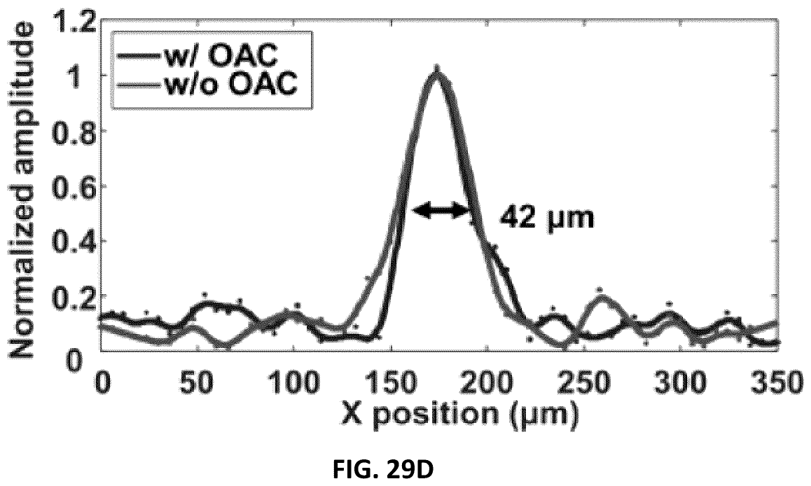

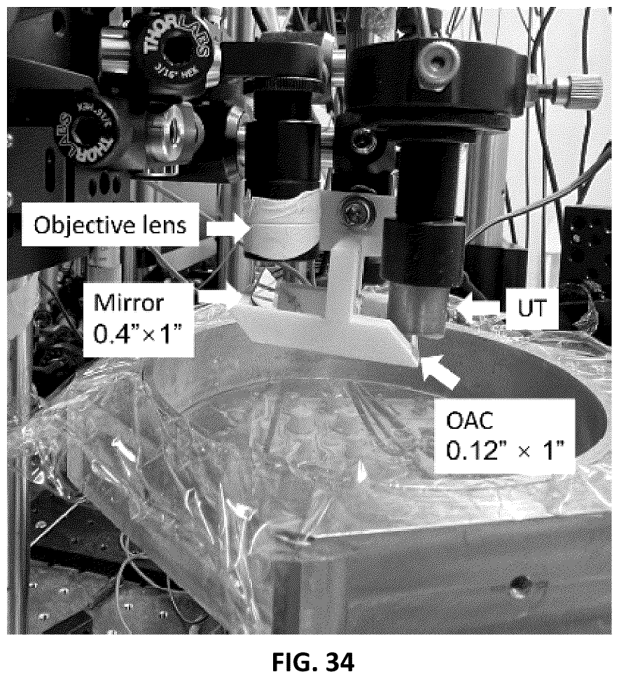

The configuration of the high-speed multi-parametric PAM system is shown in FIG. 35A. Pulses from an ns-pulsed laser (VGEN-G-20, Spectra-Physics; repetition rate, 1 MHz; wavelength, 532 nm) are directed into two different optical paths by an optical switch that consists of a half-wave plate (HWP; WPH05M-532, Thorlabs) and an electro-optical modulator (EOM; 350-Conoptics). When a low voltage is applied to the EOM, the light polarization remains unchanged. After passing through a polarizing beam splitter (PBS; PBS121, Thorlabs), the laser beam is coupled into a polarization-maintaining single-mode fiber (PM-SMF; HB450-SC, Fibercore; length, 8 m) through a fiber collimator (FC; CFC-11X-A, Thorlabs) for wavelength conversion based on the stimulated Raman scattering effect. At the fiber output, a band-pass filter (BPF; ZET561/10X, Chroma) is applied to isolate the second Stokes peak at 558 nm. If a high voltage is applied to the EOM, the light polarization is rotated by 90°. After being reflected by the PBS, the 532-nm laser beam undergoes no wavelength conversion and is directly combined with the 558-nm Stokes beam through a dichroic mirror (DM; T550lpxr, Chroma). After 5% energy is tapped off by a beam sampler (BS; BSF10-A, Thorlabs) for monitoring of the laser pulse fluctuation by a photodiode (PD; PDA25K2, Thorlabs), the dual-wavelength beam is coupled into the scanning head via a 1-m-long regular SMF (P1-460B-FC-1, Thorlabs). In the scanning head (dashed box in FIG. 29), the dual-wavelength beam is collimated by an FC (CFC-11X-A, Thorlabs), scanned by a resonant GM (6SC12KA040-04Y, Cambridge), relayed by a lens pair (L1 and L2; AC254-050-A, Thorlabs), focused by an objective lens (AC127-050-A-ML), and finally reflected by a thin metallic mirror (PFR10-P01, Thorlabs) and the OAC onto the imaging target for optical excitation. A photo of the actual scanning head is shown in FIG. 34. The scanning direction of the GM is carefully aligned to overlap the line focus of the transducer (UT; customized by the Ultrasonic Transducer Resource Center at the University of Southern California; center frequency, 35 MHz) to maximize the sensitivity of ultrasonic detection.

The scanning strategy of the high-speed wide-field PAM system is shown in FIG. 29B. The resonant GM steers the light focus within the line-shaped focus of the transducer at 12 kHz. When the laser runs at its maximum pulse repetition rate of 1 MHz, 83 A-lines are acquired across the 4.5-mm line focus in each round trip of the GM scanner. To achieve a reasonably small step size (i.e., 10 μm) along this scanning direction, every 12 lines are grouped. In each group, the next line of laser triggers for the sampling points is delayed by a constant time in the field programmable gate array (FPGA) controller to generate the 10-μm displacement from the corresponding points in the current line. Thus, by combining the 12 lines, a step size of 10 μm can be achieved across the 4.5-mm optical scanning range. This strategy enables the simultaneous acquisition of 498 B-scans with a single mechanical scan. Given the 12-kHz round-trip scanning rate and the grouping of 12 adjacent lines, the equivalent line rate is 2 kHz, which meets the sampling density required by the correlation-based flow measurement. With the much-extended optical scanning range offered by the cylindrically focused transducer, the imaging speed of multi-parametric PAM is improved by 112 times over our original development with a spherically focused transducer. For the sO₂ measurement, the excitation wavelength is switched between 532 nm and 558 nm to distinguish between oxyhemoglobin and deoxyhemoglobin based on their optical absorption spectra. Specifically, after the acquisition of 36 lines in the optical scanning direction, a high voltage is applied to the EOM to generate 558-nm Stokes pulses for the acquisition of the next 12 lines. Two spatially adjacent A-lines acquired at the two wavelengths are used for the sO₂ calculation.

To further examine the influence of the OAC on PAM's resolution, the same resolution target (R1DS1P, Thorlabs) was imaged using both reflection-mode PAM (with the OAC) and transmission-mode PAM (without the OAC). As shown in FIG. 29C, the lateral resolution was experimentally measured to be ˜6.9 μm with or without the OAC, which indicates that the OAC has good flatness and does not impair the beam quality. To achieve high temporal resolution, the lateral resolution along the fast-scanning direction is practically limited to the step size. The axial resolution, which is determined by the bandwidth of the generated ultrasonic signal, was experimentally measured to be ˜42 μm with or without the OAC, by analyzing the Hilbert transform of a representative A-line signal acquired from the resolution target [FIG. 29D]. Together, these results suggest that the OAC does not adversely affect the spatial resolution of PAM.

After developing the high-speed wide-field multi-parametric PAM in reflection mode and benchmarking its performance in standard settings, the in vivo performance was further tested in awake mouse brains (CD-1, Male, 12 weeks, Charles River). After craniotomy, a 3D-printed metal frame was installed on the mouse head by following our previously developed cranial window technique. The mouse head was secured in a head-restraint setting beneath the PAM apparatuses during imaging. The pulse energy at the surface of the brain was ˜120 nJ. All animal procedures were carried out in conformity with a laboratory animal protocol approved by the Animal Care and Use Committee at Washington University in St. Louis.

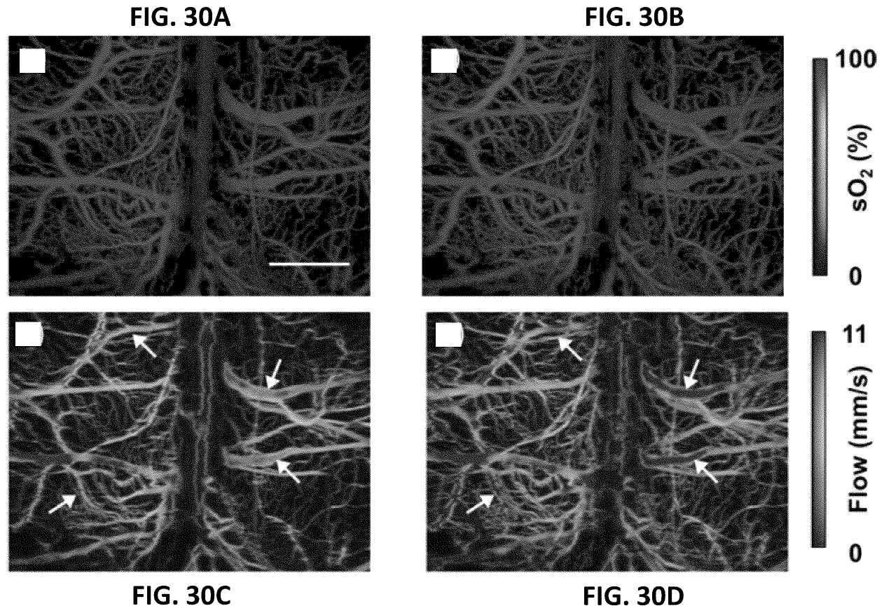

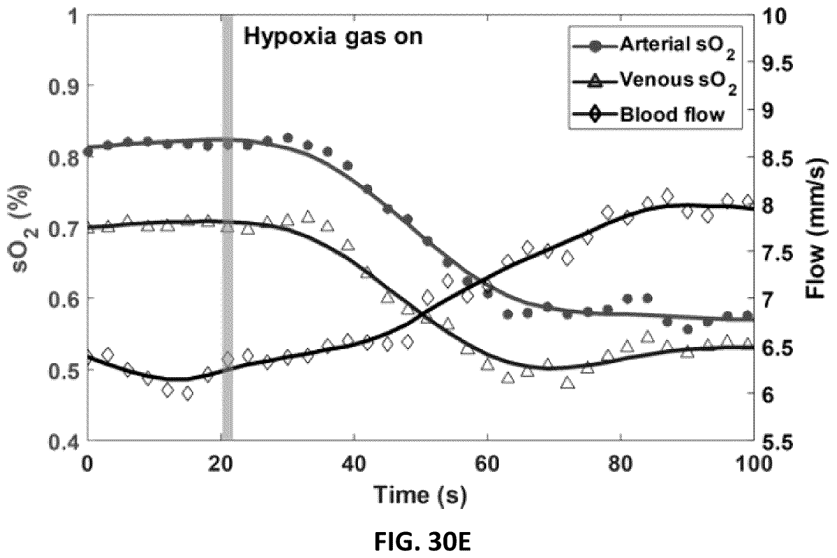

First, the system was tested in the multi-parametric imaging mode, where dense sampling was used for the simultaneous acquisition of both sO₂ and blood flow speed over an area of 4.5×3 mm² at a frame rate of 0.3 Hz. After acquiring the baseline sO₂ and blood flow under normoxia for 20 s, the inhalation gas was switched from medical air to hypoxic gas (10% oxygen), and the mouse was monitored for another 80 s. The relatively high sO₂ and flow speed of the superior sagittal sinus in FIG. 30 is inaccurate and likely due to the out-of-focus issue. The high speed of the multi-parametric PAM reveals the dynamic responses of the cortical vessels to the hypoxic challenge. Side-by-side comparison of the two representative image sets acquired under normoxia and hypoxia [FIGS. 30A and 30C, and FIGS. 30B and 30D, respectively] shows that the hypoxic challenge resulted in a marked decrease in the sO₂ and an attendant increase in the blood flow speed. These results echo our previous observations using a low-speed multi-parametric PAM system which, however, could not provide the dynamic insights shown in FIG. 30E.

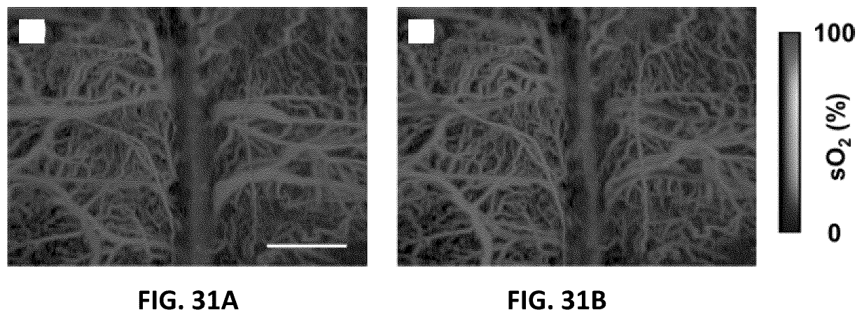

In cases where blood flow measurement is not of interest, the speed of the system can be further increased by six times. To demonstrate this ability, the hypoxia experiment was repeated under the mode of sO₂ acquisition only. A cortical area of 4.5×3 mm² was monitored at a frame rate of 2 Hz for 40 s, during which the inhalation gas was switched from medical air to the hypoxic gas with 10% oxygen. Again, the representative sO₂ images acquired under normoxia and hypoxia [FIGS. 31A and 31B, respectively] show that the sO₂ decreases in response to hypoxia. Further, the high-speed monitoring reveals the dynamic changes in the sO₂.

In summary, high-speed wide-field multi-parametric PAM in reflection mode has been successfully implemented. The thin-film OAC, which reflects light while transmitting ultrasound, accommodates the short working distance of the cylindrically focused transducer— thereby enabling the integration of the rapid optical scanning across the line-shaped acoustic focus and the conventional mechanical scanning of the optical-acoustic dual foci in reflection mode for simultaneous acquisition of 498 densely sampled B-scans. Also, the resonant GM avoids the pulse waste associated with the polygon mirror (it's been reported that 40% of the laser pulses are unusable) and possible thermal drift or cutout when over-driving a non-resonant GM. Compared to previously developed multi-parametric PAM systems, this system improves the speed by 112 times, enabling simultaneous mapping of the blood oxygenation and flow over a 4.5×3 mm² area in 3 s. Compared to the state-of-the-art high-speed PAM technique alternative, this system enables the wide-field recording of blood flow dynamics besides blood oxygenation.

What is claimed is:

1. A reflection-mode, ultra-high-speed, multi-parametric photoacoustic microscopy (PAM) system, comprising:
a. a high-repetition-rate pulsed laser configured to produce laser pulses at first and second pulse wavelengths;
b. a high-speed resonant galvanometer configured to scan the laser pulses in an optical scanning pattern;
c. a cylindrically focused transducer configured to detect photoacoustic signals produced by a sample in response to the laser pulses; and
d. an optical-acoustic combiner (OAC) configured to reflect the laser pulses into the sample and to transmit the photoacoustic signals to the transducer.

2. The system of claim 1, wherein the OAC comprises a base layer, a reflecting layer formed on the base layer, and a protective layer formed on the reflecting layer opposite the base layer.

3. The system of claim 1, wherein the photoacoustic signals are ultrasound pulses.

4. The system of claim 2, wherein the base layer is acoustically matched to a coupling medium configured to acoustically couple the sample to the transducer.

5. The system of claim 4, wherein the coupling medium is water.

6. The system of claim 2, wherein the base layer comprises acrylic with a density of about 1.18 g/cm³, an acoustic velocity of about 2.8×10³ m/s, and a thickness of about 100 μm.

7. The system of claim 2, wherein the reflecting and protective layers each comprise a thickness much less than an ultrasonic wavelength of the photoacoustic signals.

8. The system of claim 7, wherein the reflecting layer comprises aluminum with a thickness of about 250 nm.

9. The system of claim 7, wherein the protective layer comprises SiO₂ with a thickness of about 190 nm.
